(19) United States
(12) Patent Application Publication
(10) Pub. No.: US 2026/0108452 A1
(43) Pub. Date: Apr. 23, 2026

(54) REGENERATIVE COMPOSITIONS AND METHODS OF MAKING AND USING THE SAME

(71) Applicant: INVO Aesthetics Inc., Wilmington, DE (US)

(72) Inventor: Nathan CONNOLLY, Philadelphia, PA (US)

(73) Assignee: INVO Aesthetics Inc., Wilmington, DE (US)

(21) Appl. No.: 19/425,868

(22) Filed: Dec. 18, 2025

Related U.S. Application Data
(60) Provisional application No. 63/738,005, filed on Dec. 23, 2024.

(51) Int. Cl. A61K 8/98; A61K 8/34; A61K 8/39; A61K 8/41; A61K 8/49; A61K 8/55; A61K 8/64; A61K 8/66; A61K 8/73; A61K 8/81; A61K 8/92; A61K 8/99; A61Q 19/00

(52) U.S. Cl. CPC A61K 8/981; A61K 8/345; A61K 8/39; A61K 8/41; A61K 8/4926; A61K 8/553; A61K 8/64; A61K 8/66; A61K 8/735; A61K 8/8111; A61K 8/8147; A61K 8/922; A61K 8/988; A61K 8/99; A61Q 19/00

(57) ABSTRACT

Provided herein are novel compositions comprising Human Mesenchymal Conditioned Media from mammalian-derived, such as human-derived, cell sources, wherein the cell sources comprise bone marrow mesenchymal cells (BM-MSCs), umbilical cord mesenchymal cells (UC-MSCs), and fibroblast cells. The compositions may be used for formulations and skincare solutions suitable for use for cosmeceutical applications.

### CROSS-REFERENCE TO RELATED APPLICATIONS

This application claims benefit of and priority from U.S. provisional patent application Ser. No. 63/738,005, filed Dec. 23, 2024.

The foregoing application, and all documents cited therein or during their prosecution (“application cited documents”) and all documents cited or referenced in the application cited documents, and all documents cited or referenced herein (“herein cited documents”), together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### TECHNICAL FIELD

Embodiments of this disclosure relate generally to novel compositions comprising Human Mesenchymal Conditioned Media obtained from mammalian-derived, such as human-derived, cell sources and uses of such compositions in cosmetic and dermatological formulations.

### BACKGROUND OF THE INVENTION

According to industry reports, the global skincare market size was valued at USD 109.71 billion in 2023 and is projected to grow from USD 115.65 billion in 2024 to USD 194.05 billion by 2032. Skincare products include creams, lotions, compositions, formulations, and powders, which improve the quality and health of the skin and provide nourishment. These products are used daily by individuals for various purposes, such as moisturizing, hydrating, and cleansing, beautification, repair and others.

In recent years, heightened concerns regarding skin care due to many factors, such as black spots, scars, dullness, sun damage, and tanning, have increased the demand for skin improvement products. Skin brightening products, toners, and scrubs are witnessing higher demand from the younger populations. In conjunction, products that provide solutions to skin issues such as wrinkles, the appearance of fine lines, or cracked heels are gaining significant traction owing to their increasing demand from aging populations. Changing lifestyles and consumer spending patterns on premium beauty care products are projected to support skincare market growth and demand for better and more effective products.

Consumers often wish to improve the appearance of skin imperfections, preferably with instantaneous results. Many consumer products and procedures devoted to hiding and reducing wrinkles, and fine lines are available: some of which are simple and inexpensive, for example, applying make-up, particularly a primer or colored foundation, to cover the skin (and thereby cover and/or fill the wrinkles and/or lines and provide a smoother look). Other procedures are more costly and involved: such as surgical face lifts and Botox® injections. Given cost concerns, many such products and procedures are unavailable to average consumers.

Although there are a large variety of products such as lotions and creams which are formulated to hydrate the skin, make it more supple (thereby reducing the appearance of wrinkles and/or lines) or even repair skin, most of these products have limitations. For example, oftentimes, such products are not scientifically or clinically tested or verified to have effectiveness. In addition, such products may lack long-lasting benefits.

Regenerative aesthetic treatments are a form of dermatological/cosmeceutical treatments that harness the body's natural healing abilities to stimulate soft tissue regeneration. This typically involves stimulating the skin with platelet-rich plasma, regenerative dermal fillers, or exosomes to generate collagen production. Current advancements in regenerative aesthetics predominantly rely on single-source stem cell technologies or individual growth factors to treat dermatological damage including skin aging or hair loss. These approaches however typically lack the efficacy and consistency required for optimal user outcomes.

What is needed are novel compositions and formulations that support improvements in the appearance and quality of skin. Ideally, such novel compositions and formulations should also provide long-term cosmetic benefits while simultaneously enhancing the visible appearance of both skin and hair. What is also needed are regenerative formulations that yield consistent and effective results for a variety of different applications. Furthermore, what is needed are novel compositions and formulations that are not only available for application by professionals but are also available for in home use by consumers. Preferably, such compositions and formulations should be created such that they are cruelty-free and paraben-free and suitable for all skin types.

### SUMMARY OF THE INVENTION

Provided herein are compositions, formulations and cosmeceuticals comprising Human Mesenchymal Conditioned Media formulations derived from multiple mammalian stem cell sources, such as human stem cell sources. The compositions, formulations and cosmeceuticals described and claimed herein are designed for dermatological applications including skin rejuvenation and hair restoration.

Provided herein are compositions, formulations and cosmeceuticals comprising novel Human Mesenchymal Conditioned Media formulations that integrate proprietary blends of multiple mammalian-derived or human-derived stem cell sources for providing superior regenerative effects. The proprietary Human Mesenchymal Conditioned Media formulation blends are clinically validated for supporting the skin's natural regenerative processes, helping to reduce the appearance of visible inflammation, improving the look of aging skin, and enhancing the appearance of thicker, fuller-looking hair. Though not wishing to be bound by the following theory, the formulations herein achieve these effects by leveraging a synergistic combination of bioactive molecules, including (but not limited to) growth factors, cytokines, and extracellular vesicles, secreted during stem cell cultivation. These components target pathways involved in cell proliferation, collagen synthesis, inflammation reduction, and hair restoration.

In an embodiment, the present disclosure relates to unique Human Mesenchymal Conditioned Media and compositions comprising the same. The Human Mesenchymal Conditioned Media is obtained from mammalian-derived or human-derived cell sources, wherein such mammalian-derived or human-derived cell sources comprise bone marrow mesenchymal cells (BM-MSCs), umbilical cord mesenchymal cells (UC-MSCs), and fibroblast cells. The BM-MSCs, UC-MSCs, and fibroblast cells are cultured under unique conditions comprising the use of proprietary growth media, wherein the proprietary growth media is specifically formulated for optimal cell proliferation and optimized for the secretion of regenerative molecules. The proprietary growth media for BM-MSCs and UC-MSCs, may comprise Dulbecco's Modified Eagle Medium-Ham's F-12 (DMEM-F12) supplemented with fetal bovine serum (FBS), growth factors (rhFGF, rhEGF, IGF-1), and other necessary components.

In an embodiment, provided herein are unique compositions comprising the novel Human Mesenchymal Conditioned Media described herein. The compositions may be used and/or incorporated into novel formulations for cosmetic use such as, but not limited to, rejuvenating/brightening formulations, or hair improvement solutions. The formulations contemplated herein may be suitable for in-office treatments and/or for use as take-home products. In addition to the Human Mesenchymal Conditioned Media, the formulations further comprise additional ingredients including but not limited to, proteins, peptides, cytokines, growth factors, exosomes, solvents, hydrocarbon oils hydration compounds, humectants, penetration enhancers, chelators, thickening polymers, mineral thickening agents, non-mineral thickening agents, skin-renewing-healing agents, anti-inflammatory agents, antioxidants, skin regeneration agents, collagen, stabilizers, redness reduction agents, anti-aging components, anti-wrinkle components, anti-free radical agents, DNA repair enzymes, growth factors, non-volatile fatty substances, inorganic pigments, organic colorants, soft focus powder, water-soluble solvents, preservatives, fragrances, or salts.

In an embodiment, the compositions and formulations of the invention may be used to address cosmetic skin concerns including, but not limited to, the appearance of wrinkles, fine lines, uneven tone, discoloration, roughness, dryness, and dull-looking skin.

### BRIEF DESCRIPTION OF FIGURES

The present disclosure is best understood from the following detailed description when read in conjunction with the accompanying drawings. It is emphasized that, according to common practice, the various features of the drawings are not necessarily to scale. On the contrary, the dimensions of the various features are arbitrarily expanded or reduced for clarity. Like reference numerals denote like features throughout specification and drawings.

FIG. 1 provides a graphic depicting results of a skin biopsy following application of the novel compositions comprising proprietary Human Mesenchymal Conditioned Media as described herein: H&E Staining: Improvement in Dermal Thickness and Collagen Production. Significant in improvement was observed as a result of using the proprietary compositions.

FIG. 2 provides a graphic depicting results of a dermatological biopsy following application of the novel compositions herein: Verhoeff-Van Gieson (VVG) Stain: Decrease in Solar Elastosis.

FIG. 3 provides a graphic depicting results of a dermatological biopsy following application of the novel compositions herein: Verhoeff-Van Gieson (VVG) Stain: Decrease in Solar Elastosis.

FIG. 4 provides a graphic depicting results of a dermatological biopsy following application of the novel compositions herein: Trichrome Staining: Improvement in Collagen Thickness, Density, and Horizontal Alignment.

FIG. 5 provides a graph showing differential gene expression under senescence conditions and treatment comprising the proprietary formulations described herein.

FIG. 6 provides a graph showing the effect of the proprietary formulations comprising the novel Human Mesenchymal Conditioned Media described herein on fibroblast and melanoma cell viability. An increase in cell viability was observed in the healthy fibroblast cells while there was no statistically significant change in cell viability with the melanoma cells.

FIG. 7 provides a graphic of RealTime-Glorn MT Cell Viability Assay overview study: The assay involves adding NANOLUC® luciferase and a cell-permeant prosubstrate, the MT Cell Viability Substrate, to cells in culture. The MT Cell Viability Substrate is reduced to a NanoLuc® substrate by metabolically active cells. The NANOLUC® substrate diffuses from cells into the surrounding culture medium and is rapidly used by NANOLUC® Enzyme to produce a luminescent signal. The signal correlates with the number of viable cells. Dead cells do not reduce the substrate and produce no signal.

FIG. 8 provides results of an antibody microarray study showing the growth factors and cytokines present in the novel Human Mesenchymal Conditioned Media of the invention.

FIG. 9 provides pictorial representation of skin repair following the use of a formulation comprising Composition B, over a period of three weeks accelerating the skin's natural recovery process following severe injury comprising deep lacerations on the palm of an individual's right hand.

### DETAILED DESCRIPTION

The following detailed description is exemplary and explanatory and is intended to provide further explanation of the present disclosure described herein. Other advantages, and novel features will be readily apparent to those skilled in the art from the following detailed description of the present disclosure. Texts and references mentioned herein are incorporated in their entirety.

For purposes of the description hereinafter, it is to be understood that the embodiments described below may assume alternative variations and embodiments. It is also to be understood that the specific compositions, formulations and/or processes described herein are exemplary and should not be considered as limiting.

In the present disclosure the singular forms “a,” “an,” and “the” include the plural reference, and reference to a particular numerical value includes at least that particular value, unless the context clearly indicates otherwise. Thus, for example, a reference to “an antibody” or “an antibody fragment” is a reference to one or more of such structures and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

The abbreviations or acronyms used in the present disclosure have corresponding meanings as those skilled in the art understand.

As used herein, the terms “patient,” “subject,” “individual” and the like are used interchangeably, and refer to any animal, or organs, tissues or cells thereof whether in vitro or in situ, amenable to the methods described herein. In certain embodiments, the patient, subject or individual is a vertebrate. In other embodiments, the patient, subject or individual is a mammal. Non-human mammals include, for example, livestock and pets, such as ovine, bovine, porcine, canine, feline, equine and murine mammals. In yet other embodiments, the patient, subject or individual is a human.

As used herein, the term “binding” typically refers to a non-covalent association between or among two or more entities, unless expressed indicated otherwise, for example, referring to covalent bonding. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

As used herein, the term “proprietary formulation(s)” (and iterations thereof) refers to novel compositions comprising Human Mesenchymal Conditioned Media obtained from the culture of multiple mammalian-derived stem cells, such as human-derived stem cells, including but not limited to, human bone marrow stem cells, umbilical cord stem cells, and/or fibroblast cells.

Provided herein are novel Human Mesenchymal Conditioned Media formulations that integrate proprietary blends of multiple human-derived stem cell sources to provide superior regenerative effects. The Human Mesenchymal Conditioned Media is formulated from a combination of mammalian, i.e. human bone marrow stem cells, umbilical cord stem cells, and fibroblast cells. Each of these sources contributes specific bioactive molecules (e.g., growth factors, cytokines, exosomes) that support dermatological rejuvenation and hair restoration. The stem cells are cultured in a controlled environment optimized for the secretion of regenerative molecules. Tissue culturing techniques used ensure enhanced potency and uniformity, leading to batch-to-batch consistency in the bioactive content of the media. The media is enriched with vital regenerative molecules, including growth factors, cytokines, and extracellular vesicles. These components are scientifically proven to aid in skin repair, inflammation reduction, hair follicle health, and protection against cellular mutations. Clinical studies have shown that the novel Human Mesenchymal Conditioned Media formulations described herein significantly enhance collagen and elastin production, reduce redness after cosmetic procedures (such as microneedling and laser resurfacing), and stimulate hair follicle activity, reducing hair loss and promoting new growth. Furthermore, the Human Mesenchymal Conditioned Media formulations of the invention comprise approximately over 1,200 individual protein molecules, including many skin-relevant proteins such as collagens and keratins as well as high expression levels of various growth factors and cytokines including but not limited to PDGF, VEGF, EGF; Nano Particle Analysis and Electron Microscopy techniques have detected over 361 billion exosomes per milliliter. The novel Human Mesenchymal Conditioned Media described herein is suitable for a variety of cosmeceutical aesthetic treatments, including topical skincare products, post-procedure solutions, and hair restoration products.

Provided herein are novel Human Mesenchymal Conditioned Media formulations that combine the regenerative properties of multiple stem cell sources to maximize the rejuvenating effects for both skin and hair applications. The formulations have consistent efficacy resulting from advanced culturing and processing techniques that ensure uniform bioactive content across production batches, leading to reliable results. Stringent proprietary methodology involving enhanced quality control and assurance, measures protein peptide markers in development. Furthermore, the novel Human Mesenchymal Conditioned Media formulations described herein have significant versatility and are applicable for facial rejuvenation, in-office post-procedure care (microneedling and laser resurfacing), as well as hair restoration, providing comprehensive treatment options for aesthetic practitioners and patients. Thorough testing and evaluation, including conducting clinical trials has demonstrated significant improvements in inflammation reduction, anti-aging effects, and hair restoration, offering a scientific foundation for the efficacy of the formulations claimed herein.

Unlike many existing cosmeceutical and regenerative products that rely on a single stem cell source, resulting in limited efficacy and inconsistent outcomes, the formulations provided herein utilize a proprietary blend of human-derived stem cell sources (bone marrow, umbilical, and fibroblast), creating a formulation based on comprehensive components. This unique combination delivers a broad spectrum of skin-relevant proteins, growth factors, cytokines, and extracellular vesicles, leading to robust, consistent, and effective results. By combining multiple stem cell sources, the regenerative benefits of the formulations are maximized, thereby enhancing the natural recovery process of the body and skin, as well as having anti-aging, and hair restoration capabilities compared to single-source products. This is exemplified by Composition A (described below) for take-home anti-aging care, and Composition B (described below) primarily for in-office application, both of which boost post-procedure healing.

Currently practiced aesthetic procedures like microneedling, laser resurfacing, and chemical peels can cause skin damage that requires effective post-procedure care to accelerate healing and reduce downtime: the formulations provided herein have been shown to enhance the appearance of the skin by supporting its natural rejuvenation processes and improving the look of visible redness, contributing to an improved post-procedure cosmetic experience. Products such as those comprising Composition B and Composition C are suitable for in-office application immediately following microneedling and laser resurfacing to help calm the appearance of the skin and enhance the visible quality of results. As a result, users may experience a more refined post-procedure appearance and greater overall satisfaction, which can encourage continued use as part of their cosmetic skincare regime.

Consumers often need to purchase and apply multiple products to address different skin or hair concerns, which can be time-consuming, costly, and decrease compliance. The formulations described herein are designed as multifunctional products that simultaneously address various skin concerns, such as reducing the appearance of fine lines and wrinkles, discoloration, and inflammation, while also promoting hair restoration. Composition A and Composition D are examples of products that deliver these diverse benefits. As a result, the inventions herein provide a streamlined, multi-benefit approach simplifying skincare and haircare routines, making it easier for patients to comply and achieve superior results with fewer products.

Currently available hair restoration products only provide modest improvements in hair density and thickness or take a long time to produce visible results. The novel formulations described and claimed herein include specific formulations of stem cell-derived growth factors and extracellular vesicles that promote hair follicle activity, reduce hair loss, and stimulate new hair growth. Composition D is used after treatments like microneedling boost these effects. As a result, the solutions provided herein result in faster and more noticeable improvements in hair density and health, offering a more effective solution to hair loss than conventional treatments.

Another limitation of currently available dermatological and skincare products is the variability in product formulation and batch quality leading to inconsistent results and safety concerns, particularly in products utilizing biological materials. The novel formulations described and claimed herein are developed using advanced culturing and production techniques that ensure batch-to-batch consistency in the bioactive content of the Human Mesenchymal Conditioned Media. The resulting reliability enhances product safety and ensures consistent, high-quality results across different applications and treatments, including in-office procedures based on products comprising Compositions A, B, C and D as described herein.

Many currently available anti-aging products have limited longevity and fail to deliver long-lasting effects, requiring continual reapplication to maintain results. The novel formulations comprising Compositions A, B, C and D described and claimed herein have been shown to promote long-term skin health by stimulating collagen and elastin production, which provides more durable anti-aging effects. For example, products comprising Composition A help maintain these long-term benefits at home. As a result, users may notice longer-lasting cosmetic benefits, including a more even-looking tone, improved visual smoothness, and an overall enhanced appearance, which can help maintain results between applications.

Conventional skincare products often focus only on surface-level improvements and do not provide protection against cellular-level damage, which is a key factor in aging and skin mutations. The novel formulations described and claimed herein have been tested on both human dermal fibroblasts and malignant melanoma cells, demonstrating a significant increase in viability of normal human dermal fibroblasts by over 20%, with no proliferative effects observed on malignant melanoma cells. This selective action supports targeted efficacy by the proprietary formulations described, in enhancing healthy cell regeneration while maintaining safety by preventing the growth of malignant cells, addressing a critical gap in competitive solutions further supporting its use in establishing and maintaining skin health without triggering unwanted cell growth, setting it apart from alternative products.

In certain embodiments, provided herein are unique Human Mesenchymal Conditioned Media and compositions comprising the same. The Human Mesenchymal Conditioned Media is obtained from mammalian-derived, such as human-derived, cell sources, wherein such cell sources comprise bone marrow mesenchymal cells (BM-MSCs), umbilical cord mesenchymal cells (UC-MSCs), and fibroblast cells. The BM-MSCs, UC-MSCs, and fibroblast cells are cultured under unique conditions comprising the use of proprietary growth media, wherein the proprietary growth media is specifically formulated for optimal cell proliferation and optimized for the secretion of regenerative molecules. The proprietary growth media for BM-MSCs and UC-MSCs, may comprise Dulbecco's Modified Eagle Medium-Ham's F-12 (DMEM-F12) supplemented with fetal bovine serum (FBS), growth factors (rhFGF, rhEGF, IGF-1), and other necessary components.

In certain embodiments, the proprietary growth media for culturing BM-MSCs may be supplemented with 5-50% fetal bovine serum (FBS), 5-25 ng/ml IGF-1, 75-150 pg/mL rhFGF basic, and 0.5-4.0 mM L-Alanyl-L-Glutamine. In certain embodiments, the proprietary growth media for culturing BM-MSCs may be supplemented with 10% fetal bovine serum (FBS), 15 ng/mL IGF-1, 125 pg/mL rhFGF basic, and 2.4 mM L-Alanyl-L-Glutamine.

In certain embodiments, the proprietary growth media for culturing UC-MSCs is supplemented with 5-50% FBS, 5-25 ng/mL rhFGF basic, 0.1-20 ng/mL rhFGF acidic, 0.1-20 ng/mL rhEGF and 0.5-4.0 mM L-Alanyl-L-Glutamine. In certain embodiments, the proprietary growth media for culturing UC-MSCs is supplemented with 4% FBS, 5 ng/mL rhFGF basic, 5 ng/mL rhFGF acidic, 5 ng/mL rhEGF and 2.4 mM L-Alanyl-L-Glutamine.

In certain embodiments, the proprietary growth media for culturing fibroblast cells is Dulbecco's Modified Eagle Medium (DMEM) with 5-50% FBS fetal bovine serum (FBS) and other necessary components. In certain embodiments, the proprietary growth media for culturing fibroblast cells is Dulbecco's Modified Eagle Medium (DMEM) with 8% fetal bovine serum (FBS) and other necessary components.

In some embodiments, Human Mesenchymal Conditioned Media from human-derived cell sources is derived from cell culture environments wherein the cells are maintained in the presence of antibiotic antimycotic solutions: the antibiotic antimycotic solution may be selected from (but not limited to) the group consisting of penicillin, streptomycin, amphotericin or hygromycin. In certain embodiments the antimycotic solution may comprise 50-200 Units/mL penicillin G, 50-200 ug/mL streptomycin and 5-50 ng/mL amphotericin; in certain embodiments the antimycotic solutions specifically comprise 100 Units/mL penicillin G, 100 ug/mL streptomycin and 25 ng/ml amphotericin. In certain embodiments, the antimycotic solution specifically comprise 1-50 ng/ml hygromycin, or 10 ng/ml hygromycin.

In an embodiment, provided herein are methods for producing Human Mesenchymal Conditioned Media from cultured cells, wherein the cultured cells comprise human-derived cell sources, wherein the human-derived cell sources comprise bone marrow mesenchymal cells (BM-MSCs), umbilical cord mesenchymal cells (UC-MSCs), and fibroblast cells, and the methods comprise the use of proprietary growth media as described above. In certain specific embodiments, the proprietary growth media for culturing BM-MSCs may be supplemented with 5-50% fetal bovine serum (FBS), 5-25 ng/ml IGF-1, 75-150 pg/mL rhFGF basic, and 0.5-4.0 mM L-Alanyl-L-Glutamine. In certain specific embodiments, the proprietary growth media for culturing UC-MSCs is supplemented with 5-50% FBS, 5-25 ng/mL rhFGF basic, 0.1-20 ng/mL rhFGF acidic, 0.1-20 ng/mL rhEGF and 0.5-4.0 mM L-Alanyl-L-Glutamine. In certain specific embodiments, proprietary growth media for culturing fibroblast cells comprises Dulbecco's Modified Eagle Medium (DMEM) with 5-50% FBS fetal bovine serum (FBS) and other necessary components.

The methods for producing Human Mesenchymal Conditioned Media from cultured media as described herein, comprise techniques including, but not limited to, regular monitoring of the cultured cells, both visually and under light microscopy, for ensuring healthy growth at optimal culture conditions; decanting supernatant into tissue culture vessels when cells reach a desired confluency (such as 70-80%) in growth flasks; and centrifuging the supernatant at desired speeds such as 750-2,000 RPM (1,500 RPM) for the requisite time 2-10 minutes to remove any cell debris, and to obtain cell-free Human Mesenchymal Conditioned Media.

In instances wherein the Human Mesenchymal Conditioned Media is required for immediate use, it may be treated with 1% Euxyl at room temperature. In instances where the Human Mesenchymal Conditioned Media is not required for immediate use, it may be treated with 3% sterile glycerol for long-term storage and then frozen −80° C. in low-temperature-resistant containers for long-term storage.

Provided herein are unique compositions comprising the novel Human Mesenchymal Conditioned Media described above and herein. The compositions may be used and/or incorporated into novel cosmetic formulations intended to rejuvenate, brighten, or revitalize the appearance of the skin, as well as to enhance the look of thicker, fuller-appearing hair. The cosmetic formulations contemplated herein may be suitable for in-office treatments and/or for use as take-home products. In addition to the Human Mesenchymal Conditioned Media, the formulations further comprise additional ingredients including but not limited to, proteins, peptides, cytokines, growth factors, exosomes, solvents, hydrocarbon oils hydration compounds, humectants, penetration enhancers, chelators, thickening polymers, mineral thickening agents, non-mineral thickening agents, skin-improving agents, anti-inflammatory agents, antioxidants, skin regeneration agents, collagen, stabilizers, redness reduction agents, anti-aging components, anti-wrinkle components, anti-free radical agents, DNA repair enzymes, growth factors, non-volatile fatty substances, inorganic pigments, organic colorants, soft focus powder, water-soluble solvents, preservatives, fragrances, or salts.

In an embodiment, provided herein is a product formulation comprising Composition A, wherein the formulation provides long-term skin rejuvenation through at-home use. It combines the benefits of the novel cultured media with hydrating agents including hyaluronic acid and niacinamide to maintain skin health and support the regeneration process. The Composition A formulation is suitable for users seeking to sustain the effects of professional treatments and achieve the appearance of firmer skin over time.

In an embodiment, the formulation comprises a Composition A, wherein the formulation comprises a high-efficacy facial serum enabling anti-aging benefits, targeting fine lines, wrinkles, skin tone, and elasticity. In certain embodiments, the Composition A Formulation comprises, Water (Aqua), Human Mesenchymal Human Mesenchymal Conditioned Media, Glycerin, Pentylene Glycol, Niacinamide, Cetyl Ethylhexanoate, Dimethyl Isosorbide, Polyacrylate-13, sh-Polypeptide-3, sh-Polypeptide-77, sh-Polypeptide-2, sr-Honey Bee Defensin-1, Palmitoyl Tripeptide-1, Tetrapeptide-14, sh-Oligopeptide-2, sh-Polypeptide-1, sh-Polypeptide-9, sh-Polypeptide-11, sh-Oligopeptide-1, Superoxide Dismutase, Carnosine, Cetyl Stearate, Polyisobutene, Polysorbate 20, Tetrahexyldecyl Ascorbate, Propanediol, Lecithin, Sodium Hyaluronate, Helianthus annuus (Sunflower) Seed Oil, Arnica montana Flower Extract, Acetyl Glutamine, Bacillus/Folic Acid Ferment Filtrate Extract, Caprylyl Glycol, Ethylhexylglycerin, Trisodium Ethylenediamine Disuccinate, Dehydroacetic Acid, Phenoxyethanol, Ammonium Sulfate, Tocopherol, Benzyl Alcohol, Citric Acid, and Sodium Hydroxide.

In certain specific embodiments, the Composition A Formulation comprises Water (Aqua) 50-70 (% w/w), Human Mesenchymal Human Mesenchymal Conditioned Media 5-25 (% w/w), Glycerin 1-10 (% w/w), Pentylene Glycol 1-5 (% w/w), Niacinamide 1-10 (% w/w), Cetyl Ethylhexanoate 0.1-10.0 (% w/w), Dimethyl Isosorbide 0.5-5.0 (% w/w), Polyacrylate-13 0.1-5 (% w/w), sr-Honey Bee Defensin-1 0.001-0.1 (% w/w), Palmitoyl Tripeptide-1 0.001-0.01 (% w/w), Tetrapeptide-14 1-10 (% w/w), sh-Oligopeptide-2 0.1-1.0 (% w/w), Polypeptides (sh-Polypeptide-77 and sh-Polypeptide-2 and Superoxide Dismutase, sh-0.001-0.1 (% w/w)), sh-Polypeptide-3 0.001-0.1 (% w/w), sh-Oligopeptide-1 0.1-1.0 (% w/w), Carnosine 0.1-1.0 (% w/w), Cetyl Stearate 0.1-5.0 (% w/w), Polyisobutene 1-5 (% w/w), Polysorbate 20 1-5 (% w/w), Tetrahexyldecyl Ascorbate 0.01-2 (% w/w), Propanediol 0.1-5 (% w/w), Lecithin 0.1-5 (% w/w), Sodium Hyaluronate 0.1-1.0 (% w/w), Helianthus annuus (Sunflower) Seed Oil 0.01-2 (% w/w), Arnica montana Flower Extract 0.01-2 (% w/w), Acetyl Glutamine 0.1-5.0 (% w/w), Bacillus/Folic Acid Ferment Filtrate Extract 0.1-1.0 (% w/w), Caprylyl Glycol 0.1-5.0 (% w/w), Ethylhexylglycerin 0.1-5.0 (% w/w), Trisodium Ethylenediamine Disuccinate 0.1-1 (% w/w), Dehydroacetic Acid 0.1-5.0 (% w/w), Phenoxyethanol 0.1-5.0 (% w/w), Ammonium Sulfate 0.1-5.0 (% w/w), Tocopherol 0.1-5.0 (% w/w), Benzyl Alcohol 0.1-10.0 (% w/w), Citric Acid 0.1-10.0 (% w/w), and Sodium Hydroxide 0.0-5.0 (% w/w).

In certain specific embodiments, the Composition A Formulation comprises:

TABLE 1
Composition A

| INGREDIENTS INCI/CTFA NAME | % W/W |
|---|---|
| Water (Aqua) | 55-65 |
| Sodium Hyaluronate | 0.200 |
| Pentylene Glycol | 2-5 |
| Trisodium Ethylenediamine Disuccinate | 0.300 |
| Water (Aqua) | |
| Niacinamide | 2-6 |
| Propanediol | 0.300 |
| Lecithin | |
| Polyacrylate-13 | 2-4 |
| Polyisobutene | |
| Polysorbate 20 | |
| Cetyl Stearate | 0.500 |
| Helianthus Annuus (Sunflower) Seed Oil | 0.020 |
| Arnica Montana Flower Extract | |
| Tetrahexyldecyl Ascorbate | 0.100 |
| Dimethyl Isosorbide | 1-4 |
| Cetyl Ethylhexanoate | 2-6 |
| Human Mesenchymal Conditioned Media | 10-15 |
| Phenoxyethanol | |
| Ethylhexylglycerin | |
| Glycerin | 4-7 |
| Tetrapeptide-14 | 4-7 |
| Water (Aqua) | 2.000 |
| Palmitoyl Tripeptide-1 | 0.001 |
| Carnosine | 0.200 |
| sh-Polypeptide-77 | 0.010 |
| sh-Polypeptide-2 | |
| Superoxide Dismutase | |
| sh -Polypeptide-3 | 0.010 |
| sr-Honey Bee Defensin-1/Royal Jelly Extract | 0.02-0.1 |
| Water | 3.000 |
| Lecithin | |
| Acetyl Glutamine | |
| Propanediol | |
| sh-Oligopeptide-1 | |
| sh-Oligopeptide-2 | |
| sh-Polypeptide-1 | |
| sh-Polypeptide-9 | |
| sh-Polypeptide-11 | |
| Bacillus/folic ferment filtrate extract | |
| Sodium hyaluronate | |
| Caprylyl glycol | |
| Ethyhexylglycerin | |
| Dehydroacetic Acid | 0.05-2 |
| Benzyl Alcohol | |
| Citric Acid | 0.1-4 |
| Sodium Hydroxide | 0.000 |

In an embodiment, provided herein is a product formulation comprising Composition B. The Composition B formulation is intended to support facial rejuvenation by enhancing the appearance of skin firmness, smoothness, and overall quality. It combines the benefits of the novel cultured media and incorporates a blend of growth factor-containing materials, including EGF (Epidermal Growth Factor) and KGF (Keratinocyte Growth Factor), which help improve the visible appearance of skin tone, texture, and radiance. The Composition B formulation is designed for use in professional in-office settings as part of post-procedure cosmetic care, complementing aesthetic treatments and enhancing the visible quality of post-procedure results.

In an embodiment, a product comprising the Composition B formulation enables skin healing and rejuvenation after cosmetic procedures such as microneedling and laser resurfacing. The Composition B formulation comprises Water (Aqua), Human Mesenchymal Human Mesenchymal Conditioned Media, Glycerin, Pentylene Glycol, Niacinamide, sh-Oligopeptide-1, sh-Polypeptide-3, sr-Honey Bee Defensin-1, Acetyl Tetrapeptide-2, Palmitoyl Hexapeptide-12, Tetrapeptide-14, sh-Oligopeptide-2, sh-Polypeptide-1, sh-Polypeptide-9, sh-Polypeptide-11, Propanediol, Lecithin, Sodium Hyaluronate, Helianthus annuus (Sunflower) Seed Oil, Arnica montana Flower Extract, Tasmannia lanceolata Fruit/Leaf Extract, Acetyl Glutamine, Bacillus/Folic Acid Ferment Filtrate Extract, Caprylyl Glycol, Ethylhexylglycerin, Trisodium Ethylenediamine Disuccinate, Dehydroacetic Acid, Phenoxyethanol, Ammonium Sulfate, Tocopherol, Benzyl Alcohol, Citric Acid, Sodium Hydroxide.

In certain specific embodiments, the Composition B formulation comprises Water (Aqua) 40-80 (% w/w), Human Mesenchymal Human Mesenchymal Conditioned Media 5-30 (% w/w), Glycerin 0.1-15 (% w/w), Pentylene Glycol 1-10 (% w/w), Niacinamide 1-15 (% w/w), sh-Oligopeptide-1 0.1-10 (% w/w), sh-Polypeptide-3 0.001-1 (% w/w), sr-Honey Bee Defensin-1 0.001-1 (% w/w), Acetyl Tetrapeptide-2 0.001-5 (% w/w), Palmitoyl Hexapeptide-12 0.001-1 (% w/w), Tetrapeptide-14 0.001-1 (% w/w), sh-Oligopeptide-2 0.001-5 (% w/w), sh-Polypeptide-1 0.001-5 (% w/w), sh-Polypeptide-9 0.001-5 (% w/w), sh-Polypeptide-11 0.001-5 (% w/w), Propanediol 0.001-5 (% w/w), Lecithin 0.001-5 (% w/w), Sodium Hyaluronate 0.001-5 (% w/w), Helianthus annuus (Sunflower) Seed Oil 0.001-5 (% w/w), Arnica montana Flower Extract 0.001-5 (% w/w), Tasmannia lanceolata Fruit/Leaf Extract 0.001-5 (% w/w), Acetyl Glutamine 0.001-5 (% w/w), Bacillus/Folic Acid Ferment Filtrate Extract 0.001-5 (% w/w), Caprylyl Glycol 0.001-5 (% w/w), Ethylhexylglycerin 0.001-5 (% w/w), Trisodium Ethylenediamine Disuccinate 0.001-5 (% w/w), Dehydroacetic Acid 0.001-5 (% w/w), Phenoxyethanol 0.001-20 (% w/w), Ammonium Sulfate 0.001-10 (% w/w), Tocopherol 0.001-10 (% w/w), Benzyl Alcohol 0.001-10 (% w/w), Citric Acid 0.1-10.0 (% w/w), and Sodium Hydroxide 0.0-5.0 (% w/w).

In certain specific embodiments, products comprising Composition B formulation comprise:

TABLE 2
Composition B

| INGREDIENTS INCI/CTFA NAME | % W/W |
|---|---|
| Water (Aqua) | 58-72 |
| Sodium Hyaluronate | 0.1500 |
| Glycerin | 0.1-1 |
| Propanediol | 0.01-2.0 |
| Lecithin | |
| Helianthus Annuus (Sunflower) Seed | 0.01-0.1 |
| Oil | |
| Arnica Montana Flower Extract | |
| Trisodium Ethylenediamine | 0.1-0.5 |
| Disuccinate | |
| Water (Aqua) | |
| Dehydroacetic Acid | 0.1-2.0 |
| Benzyl Alcohol | |
| Pentylene Glycol | 2.000-6.000 |
| Niacinamide | 1.000-5.000 |
| sh-Oligopeptide-1 | 0.01-1.000 |
| sh -Polypeptide-3 | 0.0100-1.000 |
| sr-Honey Bee Defensin-1/Royal Jelly | 0.01-0.0800 |
| Extract | |
| Palmitoyl Hexapeptide | 0.0010-0.0050 |
| Acetyl Tetrapeptide-2 | 0.0050 |
| Water | 1-5.000 |
| Lecithin | |
| Acetyl Glutamine | |
| Propanediol | |
| sh-Oligopeptide-1 | |
| sh-Oligopeptide-2 | |
| sh-Polypeptide-1 | |
| sh-Polypeptide-9 | |
| sh-Polypeptide-11 | |
| Bacillus/folic ferment filtrate extract | |
| Sodium hyaluronate | |
| Caprylyl glycol | |
| Ethyhexylglycerin | |
| Glycerin | 3.000-6.000 |
| Tetrapeptide-14 | |
| Glycerin | 1.000-4.000 |
| Water | |
| Tasmania Lanceolata Fruit Leaf extract | |
| Human Mesenchymal Conditioned | 10.000-20.000 |
| Media | |
| Phenoxyethanol | |
| Ethylhexylglycerin | |
| Citric Acid | 0.1-0.2 |

In an embodiment, provided herein is a product formulation comprising Composition C, wherein the Composition C is formulated to target and improve uneven skin tone, alleviate sun damage, and reduce discoloration. Composition C utilizes ingredients such as tranexamic acid and niacinamide alongside the novel Human Mesenchymal Conditioned Media, growth factors and peptides. The combination aids in reducing dark spots and enhancing overall skin radiance, offering a non-invasive solution for skin brightening. Products comprising Composition C enable deep penetration of active ingredients, making them suitable for both professional treatments and at-home maintenance routines.

In an embodiment, provided herein are formulations comprising Composition C, wherein Composition C comprises growth factors, cytokines, and exosomes to promote collagen production, reduce inflammation, and accelerate skin recovery. The products and formulations comprising Composition C also address pigmentation issues, reduce discoloration, improve skin tone, reduce uneven skin tone, and alleviate sun damage. Though not wishing to be bound by the following theory, the unique formulations comprising Composition C enable the modification of melanin production while enhancing skin clarity and brightness. In certain embodiments, Composition C and formulations thereof, comprise Water (Aqua), Human Mesenchymal Human Mesenchymal Conditioned Media, Tranexamic Acid, Glycerin, Pentylene Glycol, Niacinamide, Acetyl Glycyl Beta-Alanine, 1,2-Hexanediol, Pentasodium Tetracarboxymethyl Acetylhydroxyprolyl Dipeptide-12, Pentasodium Tetracarboxymethyl Dipeptide-51, Tetrapeptide-14, sr-Honey Bee Defensin-1, Propanediol, Lecithin, Sodium Hyaluronate, Helianthus annuus (Sunflower) Seed Oil, Arnica montana Flower Extract, Tasmannia lanceolata Fruit/Leaf Extract, Ethylhexylglycerin, Trisodium Ethylenediamine Disuccinate, Dehydroacetic Acid, Phenoxyethanol, Ammonium Sulfate, Tocopherol, Benzyl Alcohol, Citric Acid, Sodium Hydroxide.

In certain specific embodiments, Composition C comprises Water (Aqua) 40-80 (% w/w), Human Mesenchymal Human Mesenchymal Conditioned Media 5-30 (% w/w), Tranexamic Acid 1-15 (% w/w), Glycerin 0.001-5 (% w/w), Pentylene Glycol 0.01-15 (% w/w), Niacinamide 0.1-10 (% w/w), Acetyl Glycyl Beta-Alanine 0.1-10 (% w/w), 1,2-Hexanediol 0.001-5 (% w/w), Pentasodium Tetracarboxymethyl Acetylhydroxyprolyl Dipeptide-12 0.001-5 (% w/w), Pentasodium Tetracarboxymethyl Dipeptide-51 0.001-5 (% w/w), Tetrapeptide-14 0.001-5 (% w/w), sr-Honey Bee Defensin-1 0.001-5 (% w/w), Propanediol 0.001-5 (% w/w), Lecithin 0.001-5 (% w/w), Sodium Hyaluronate 0.001-5 (% w/w), Helianthus Annuus (Sunflower) Seed Oil 0.001-5 (% w/w), Arnica montana Flower Extract 0.001-5 (% w/w), Tasmannia lanceolata Fruit/Leaf Extract 0.001-5 (% w/w), Ethylhexylglycerin 0.1-25 (% w/w), Trisodium Ethylenediamine Disuccinate 0.001-5 (% w/w), Dehydroacetic Acid 0.001-5 (% w/w), Phenoxyethanol 0.001-20 (% w/w), Ammonium Sulfate 0.001-10 (% w/w), Tocopherol 0.001-10 (% w/w), Benzyl Alcohol 0.001-10 (% w/w), Citric Acid 0.1-10.0 (% w/w), and Sodium Hydroxide 0.0-5.0 (% w/w).

In certain specific embodiments, products comprising Composition C formulations comprise:

TABLE 3
Composition C

| INGREDIENTS INCI/CTFA NAME | % W/W |
|---|---|
| Water (Aqua) | 50-60 |
| Glycerin | 0.5-2.5 |
| Sodium Hyaluronate | 0.05-1.00 |
| Propanediol | 0.01-1.00 |
| Lecithin | |
| Helianthus Annuus (Sunflower) Seed Oil Arnica | 0.01-1.00 |
| Montana Flower Extract | |
| Trisodium Ethylenediamine Disuccinate | 0.01-1.00 |
| Dehydroacetic Acid | 0.5-2.00 |
| Benzyl Alcohol | |
| Pentylene Glycol | 1.000-8.000 |
| Tranexamic Acid | 4.00010.000 |
| Niacinamide | 1.000-5.000 |
| Acetyl Glycyl Beta-Alanine | 0.5-3.000 |
| Water (Aqua) | 1.000-3.000 |
| 1,2-Hexanediol | |
| Pentasodium Tetracarboxymethyl Dipeptide-51 | |
| Pentasodium Tetracarboxymethyl | |
| Acetylhydroxyprolyl Dipeptide-12 | |
| sr-Honey Bee Defensin-1 | 0.005-1.000 |
| Royal Jelly Extract | |
| Glycerin | 1.000-3.000 |
| Water | |
| Tasmannia Lanceolata Fruit/Leaf Extract | |
| Glycerin | 2.000-7.000 |
| Tetrapeptide-14 | |
| Human Mesenchymal Conditioned Media | 10.000-20.000 |
| Phenoxyethanol | |
| Ethylhexylglycerin | |
| Citric Acid | 0.000 |
| Sodium Hydroxide | 0.000 |

In an embodiment, provided herein is a product formulation comprising Composition D, wherein the product formulation is aimed at stimulating hair growth and improving scalp health. Composition D formulations comprise Keratinocyte Growth Factor (KGF) to promote the growth of hair follicles and strengthen existing strands. The product is designed for in-office use, providing a comprehensive approach to hair restoration by boosting scalp vitality and encouraging thicker, fuller hair.

In an embodiment, the formulation comprises Composition D, wherein the formulation comprising Composition D stimulates hair follicles and promotes hair restoration, reduces thinning hair, reduces hair loss, and improves scalp health. In an embodiment, Composition D comprises Water (Aqua), Human Mesenchymal Human Mesenchymal Conditioned Media, Pentylene Glycol, Glycerin, Caffeine, 1,2-Hexanediol, Acetyl Tripeptide-54 Amide, Acetyl sh-Tripeptide-4 Amide, Tripeptide-80, sh-Polypeptide-3, Acetyl Glutamine, sh-Oligopeptide-1, sh-Oligopeptide-2, sh-Polypeptide-1, sh-Polypeptide-9, sh-Polypeptide-11, Propanediol, Lecithin, Sodium Hyaluronate, Helianthus annuus (Sunflower) Seed Oil, Nasturtium officinale (Watercress) Extract, Tropaeolum majus (Garden Nasturtium) Extract, Sorbic Acid, Potassium Azeloyl Diglycinate, Extract, Serenoa repens (Saw Palmetto) Berry Extract, Bacillus/Folic Acid Ferment Filtrate Extract, Caprylyl Glycol, Ethylhexylglycerin, Trisodium Ethylenediamine Disuccinate, Dehydroacetic Acid, Phenoxyethanol, Tocopherol, Benzyl Alcohol, Citric Acid, Sodium Hydroxide.

In a specific embodiment, Composition D comprises Water (Aqua) 40-80 (% w/w), Human Mesenchymal Human Mesenchymal Conditioned Media 5-25 (% w/w), Pentylene Glycol 0.1-10 (% w/w), Glycerin 0.1-5 (% w/w), Caffeine 0.1-10 (% w/w), 1,2-Hexanediol 0.01-20 (% w/w), Acetyl Tripeptide-54 Amide 0.001-25 (% w/w), Acetyl sh-Tripeptide-4 Amide 0.001-25 (% w/w), Tripeptide-80 0.001-25 (% w/w), sh-Polypeptide-3 0.001-25 (% w/w), Acetyl Glutamine 0.001-10 (% w/w), sh-Oligopeptide-1 0.001-10 (% w/w), sh-Oligopeptide-2 0.001-10 (% w/w), sh-Polypeptide-1 0.001-10 (% w/w), sh-Polypeptide-9 0.001-10 (% w/w), sh-Polypeptide-11 0.001-10 (% w/w), Propanediol 0.001-10 (% w/w), Lecithin 0.001-10 (% w/w), Sodium Hyaluronate 0.001-10 (% w/w), Helianthus annuus (Sunflower) Seed Oil 0.001-5 (% w/w), Nasturtium officinale (Watercress) Extract 0.001-5 (% w/w), Tropaeolum majus (Garden Nasturtium) Extract 0.001-5 (% w/w), Sorbic Acid 0.001-10 (% w/w), Potassium Azeloyl Diglycinate 0.001-5 (% w/w), Extract, Serenoa repens (Saw Palmetto) Berry Extract 0.001-5 (% w/w), Bacillus/Folic Acid Ferment Filtrate Extract 0.001-5 (% w/w), Caprylyl Glycol 0.001-5 (% w/w), Ethylhexylglycerin 0.1-25 (% w/w), Trisodium Ethylenediamine Disuccinate 0.001-5 (% w/w), Dehydroacetic Acid 0.001-5 (% w/w), Phenoxyethanol 0.001-20 (% w/w), Tocopherol 0.001-10 (% w/w), Benzyl Alcohol 0.001-10 (% w/w), Citric Acid 0.1-10.0 (% w/w), and Sodium Hydroxide 0.0-5.0 (% w/w).

In a specific embodiment, the Composition D Formulation comprises:

TABLE 4
Composition D

| INGREDIENTS INCI/CTFA NAME | % W/W |
|---|---|
| Water (Aqua) | 40.0-60.0 |
| Sodium Hyaluronate | 0.01-0.40 |
| Propanediol & Lecithin | 0.05-0.5 |
| Trisodium Ethylenediamine & Disuccinate | 0.100-0.5000 |
| Dehydroacetic Acid & Benzyl Alcohol | 0.500-2.000 |
| Pentylene Glycol | 2.000-5.000 |
| Caffeine | 0.5000-2.000 |
| Glycerin w/Water (Aqua) & | 0.5-2.000 |
| Serenoa Serrulata Fruit Extract | |
| sh-polypeptide-3 | 0.0100-0.1000 |
| Glycerin w/Water (Aqua) & | 0.1000-1.000 |
| Nasturtium officinale extract & | |
| Tropaeolum majus extract | |
| Water | 1.000-5.000 |
| Lecithin | |
| Acetyl Glutamine | |
| Propanediol | |
| sh-Oligopeptide-1 | |
| sh-Oligopeptide-2 | |
| sh-Polypeptide-1 | |
| sh-Polypeptide-9 | |
| sh-Polypeptide-11 | |
| Bacillus/folic acid ferment filtrate extract | |
| Sodium hyaluronate | |
| Caprylyl glycol | |
| Ethylhexylglycerin | |
| Water | 5.000-15.0000 |
| 1,2-Hexanediol | |
| Caprylyl Glycol | |
| Tripeptide-80 | |
| Acetyl Tetrapeptide-54 Amide | |
| Water | 5.000-15.0000 |
| 1,2 Hexanediol | |
| Caprylyl Glycol | |
| Tripeptide-80 | |
| Acetyl sh Tripeptide-4 Amide | |
| Human Mesenchymal Conditioned Media | 10.000-20.000 |
| Phenoxyethanol | |
| Ethylhexylglycerin | |
| Potassium Azeloyl Diglycinate | 1.0000-5.0000 |
| Water | |
| Sodium Hydroxide | 0.05-0.20 |
| Citric Acid | 0.10-0.50 |

In further embodiments, included herein are methods for improving dermatological conditions comprising: using novel formulations as described above, consisting of Compositions A, B, C or D, wherein the formulations comprise novel Human Mesenchymal Conditioned Media, wherein the Human Mesenchymal Conditioned Media is obtained from human-derived cell sources comprising BM-MSCs, UC-MSCs, and fibroblast cells, wherein the BM-MSCs, UC-MSCs, and fibroblast cells are cultured under unique conditions comprising the use of proprietary growth media, wherein the proprietary growth media is specifically formulated for optimal cell proliferation and optimized for the secretion of regenerative molecules, monitoring the dermatological condition for improvement.

The formulations and skincare solutions as contemplated may comprise additional ingredients including but not limited to, cytokines, growth factors, exosomes, solvents, hydrocarbon oils hydration compounds, humectants, penetration enhancers, chelators, thickening polymers, mineral thickening agents, non-mineral thickening agents, wound-healing agents, anti-inflammatory agents, antioxidants, skin regeneration agents, collagen, stabilizers, redness reduction agents, anti-aging components, anti-wrinkle components, anti-free radical agents, DNA repair enzymes, growth factors, non-volatile fatty substances, inorganic pigments, organic colorants, soft focus powder, water-soluble solvents, preservatives, fragrances, or salts.

In certain embodiments, the formulations and skincare solutions described herein are effective for addressing skin conditions including, but not limited to, the appearance of wrinkles and fine lines, uneven tone, discoloration, roughness, dryness, and dull-looking skin. The formulations and skin solutions described herein are suitable for in-office treatments and take-home products, suitable for topical application, suitable for micro-needling application and/or intradermal application.

In certain embodiments, the formulations and skincare solutions described herein may comprise one or more additional active agents including, but not limited to: pharmaceutical agents, immunomodulatory agents, cytotoxic agents, enzymes, proteins, antimicrobial agents, interferons, vitamins, nucleic acids, steroids, aminoglycosides, antioxidants, stem cells, anesthetic agents, analgesics, anti-inflammatoires, or hormonal agents.

In certain embodiments, the formulations and skincare solutions described herein may be used to deliver cosmetic agents intended to improve the visible appearance and quality of the skin. Such improvements may include enhancing overall radiance and smoothness, refining the appearance of uneven tone or texture, and addressing the visible effects of environmental exposure. In certain embodiments, the formulations and skin solutions of the invention may be conjugated or otherwise linked with select agents that provide positive effects in aspects of skin care such as moisturizing, sunscreen, spot removal, discoloration, antiaging or other pigmentation enhancements. As used herein, the term “cosmeceutical” includes agents, substances, pharmaceuticals useful for the enhancement of the health and beauty of skin. Representative cosmeceuticals include, but are not limited to vitamins (Vitamins A or retinoids tretinoin, adapalene, tazarotene, retinaldehyde, retinol, and retinyl esters; Vitamins B (B3 nicotinamide or niacinamide), C (L-ascorbic acid), D, and E (alpha-tocopherol)), hydroxyacids (α-hydroxyacids, β-hydroxyacids, polyhydroxyacids, α-hydroxyacid glycolic acid, and bionic acids, peptides (including peptide fragments of collagen and elastin, pal-KTTKS (Matrixyl), Ac-EEMQRR (Argireline), and Cu-GHK), growth factors, botanicals, selenium, lycopene, pycnogenol zinc and copper. The constructs of the invention may be used to effectively deliver therapeutic and/or cosmeceutical agents to the cells of the epidermis (including keratinocytes, melanocytes, and Langerhans and Merkel cells), dermis (fibroblasts) or subcutitis.

The formulations and skincare solutions described herein may be utilized over an extended period of time, for example, 1-7 days, 1-20 weeks, 1-12 weeks, 1-10 weeks, 1-8 weeks, 1-6 weeks, 1-4 weeks and/or 1-2 weeks 1-52 weeks, including intervals therebetween or in perpetuity. The formulations and skin solutions may be specifically engineered to enable specific rates of release depending on the condition to be treated and other such factors.

The formulations and skincare solutions described herein may be administered as single or multiple applications and the applications may be administered as needed, for example, they may be administered twice daily, daily, every other day, every third day, three times per week, twice per week, weekly, biweekly, monthly, or bimonthly including intervals therebetween or other schedule. The application amount may be determined based on factors known to those skilled in the art (such as effective profiles and conditions to be treated) and may vary from about 1 μg/mL to about 10 μg/mL, about 10 μg/mL to about 50 μg/mL, about 50 μg/mL to about 150 μg/mL, about 150 μg/mL to about 250 μg/mL, about 250 μg/mL to about 500 μg/mL, about 500 μg/mL to about 750 μg/mL, about 750 μg/mL to about 1 mg/mL, about 1 mg/mL to about 50 mg/mL, about 1 mg/mL to about 100 mg/mL, about 50 mg/mL to about 100 mg/mL.

In certain embodiments, the administration or application of the formulations or skincare solutions of the invention improves the conditions of a subject requiring improvement in conditions such as the appearance of wrinkles and fine lines, uneven tone, discoloration, roughness, dryness, and dull-looking skin.

The novel cosmetic formulations or skincare solutions described herein may be administered by standard routes. In general, they may be administered by the topical, transdermal, or parenteral (e.g., intravenous, subcutaneous) topical administration routes. In certain embodiments, the formulations are applied via micro-needling.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Suitable application amounts or concentrations of the compositions may vary depending on the desired cosmetic effect, the condition or state being addressed and other factors such as weight and severity of condition and the route of administration of the compound. It is to be understood that the invention has application for both human, veterinary, and general animal use. The methods of the invention contemplate single as well as multiple administrations, given either simultaneously or over an extended period of time.

Preferred unit dosage formulations are those containing a daily dose or unit, daily sub-dose, as herein above recited, or an appropriate fraction thereof, of the administered ingredient. It should be understood that in addition to the ingredients, particularly mentioned above, the formulations of the present invention may include other agents conventional in the art having regard to the type of formulation in question.

The following examples are given to illustrate exemplary embodiments of the present disclosure. It should be understood, however, that the present disclosure is not to be limited to the specific conditions or details described in these examples. Examples are provided below to facilitate a more complete understanding of the invention. The following examples illustrate the exemplary modes of making and practicing the invention.

### EXAMPLES

### Example 1

### Synthesis Process & Product Formulation for Proprietary Human Mesenchymal Conditioned Media

### 1. Cell Line Acquisition:

The process begins with sourcing specific human-derived cell lines, including bone marrow mesenchymal stem cells (BM-MSC), umbilical cord mesenchymal stem cells (UM-MSC), and fibroblasts (FB). These cell lines are acquired from accredited sources and undergo strict quality control to ensure they meet the necessary standards for purity and viability.
After securing early passage cryopreserved cell stocks, specific tissue culture protocols were developed for each cell type for rapid cell expansion toward future, large volume order demands.

### 2. Cell Culturing and Expansion:

Each cell type is cultured separately using proprietary growth media specifically formulated for optimal cell proliferation. For instance, BM-MSCs and UM-MSCs are cultured in Dulbecco's Modified Eagle Medium-Ham's F-12 (DMEM-F12) supplemented with fetal bovine serum (FBS), growth factors (rhFGF, rhEGF, IGF-1), and other necessary components.
The cells are grown in vent cap flasks under controlled conditions (37° C., 5% CO2) and monitored regularly until they reach 70-80% confluency.
Primary growth media for both BM-MSC and UC-MSC is Dulbecco's Modified Eagle Medium-Ham's F-12 (DMEM-F12). BM-MSC growth is supplemented with 10% fetal bovine serum (FBS), 15 ng/mL IGF-1, 125 pg/mL rhFGF basic, and 2.4 mM L-Alanyl-L-Glutamine. UC-MSC growth is supplemented with 4% FBS, 5 ng/mL rhFGF basic, 5 ng/mL rhFGF acidic, 5 ng/mL rhEGF and 2.4 mM L-Alanyl-L-Glutamine. Cells are maintained in the presence of antibiotic antimycotic solution (100 Units/mL penicillin G, 100 ug/mL streptomycin and 25 ng/mL Amphotericin), to prevent microbial contamination. Cells are plated in the 185 mm2 vent cap flasks and are submerged in about 40 mL growth media. All cell types should grow tightly adherent to bottom side of the flasks which are placed inside a tissue culture incubator at 37° C., 5% CO2.
Daily monitoring both visually and under light microscopy ensures healthy growth at optimal culture conditions. Growing cells consume the nutrients provided in their respective growth media while releasing the protein molecules generated inside the cells back to the liquid phase media. When the cells accomplish 70-80% confluency in the flasks, the liquid media is carefully decanted into appropriate tissue culture plastic wares. The remaining cells adherent at the bottom of the flasks are washed with 1×PBS followed by 3 mL trypsin for 3 min. to detach from the flask and separate to single cells. About 1/10- 1/20 of the floating cell contents are transferred to a new flask with proper fresh growth media for next pass cell growth. The remaining majority of floating cells in the flask are discarded appropriately.

### 3. Harvesting Human Mesenchymal Conditioned Media:

Once the cells have proliferated sufficiently, the media is carefully collected by decanting the supernatant, which contains secreted proteins, growth factors, cytokines, and extracellular vesicles. This supernatant is then centrifuged at 1,500 RPM for 5 minutes to remove any cell debris, leaving cell-free Human Mesenchymal Conditioned Media rich in bioactive compounds.
The liquid media thus harvested is centrifuged at 1,500 RPM, 5 min. to discard any unwanted cell debris. The resulting media is cell-free. The Human Mesenchymal Conditioned Media thus prepared from respective cell types can be kept separately or blended at desired ratios suitable for desired applications. Fresh media is added with 1% Euxyl at room temperature for immediate use. For longer term storage, the collected media is added with 3% (w/v) sterile glycerol and placed at −80° C. freezer in durable (low-temperature resistant) plastic bottles. Upon ready to use, the frozen media is placed at 4° C. for slow thawing.

### 4. Blending Cell Sources:

The harvested Human Mesenchymal Conditioned Media from each cell type is blended in a proprietary ratio (40-50% BM-MSC, 35-45% FB, and 10-20% UM-MSC). This specific blend is designed to maximize the regenerative properties by providing a wide spectrum of bioactive molecules, including over 1,200 identified proteins, growth factors and cytokines whose concentrations are measured, and over 361 billion exosomes per milliliter.

### 5. Stabilization and Storage:

To preserve the sterility of the Human Mesenchymal Conditioned Media, it may be treated with 1% Euxyl at room temperature for immediate use or with 3% sterile glycerol for long-term storage. To preserve the bioactivity of the media, it is then frozen at −80° C. in low-temperature-resistant containers.

### 6. Final Product Formulation:

After the blending process, the Human Mesenchymal Conditioned Media is incorporated into the final product formulations. These formulations comprising Compositions A, B, C or D are optimized for use in both in-office treatments and take-home products. Stability testing and safety verification ensure that the products maintain efficacy and consistency across all batches.

### 7. Quality Control and Testing:

The final Human Mesenchymal Conditioned Media undergoes rigorous quality control and assurance measures through protein peptide markers to ensure each batch is statistically the same. Additionally, independent third-party testing is conducted on the final product formulations to ensure the products meet safety and efficacy standards.

### Example 2

### Biopsy Analysis: Dermal Regeneration and Structural Enhancement

Biopsy analysis was performed to assess the effect of the novel formulations described herein on dermal regeneration and structural enhancement.

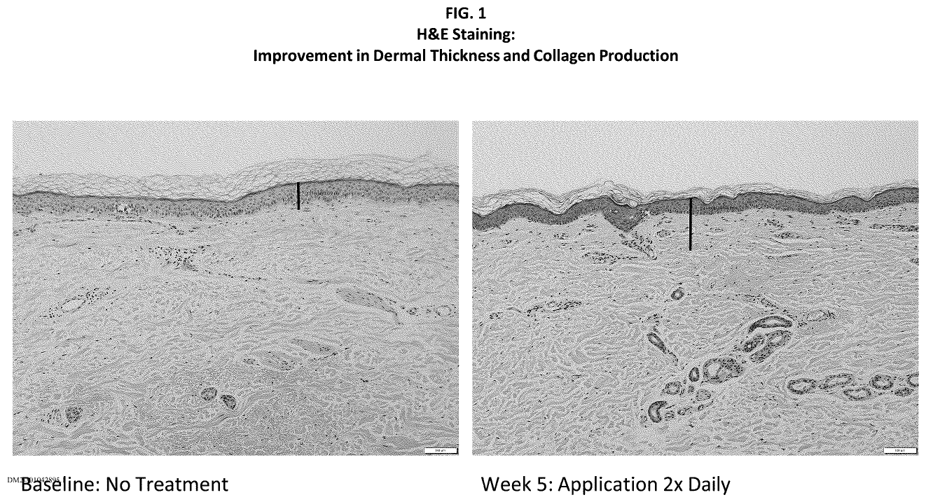

Histological Findings Biopsies at five weeks post-treatment with Composition A formulation (described above) showed a notable increase in papillary dermal thickness, as observed through hematoxylin and eosin (H&E) staining. This result indicates enhanced dermal cell proliferation and extracellular matrix (ECM) synthesis, driven by Composition A potent growth factors and exosomes. (See FIG. 1)

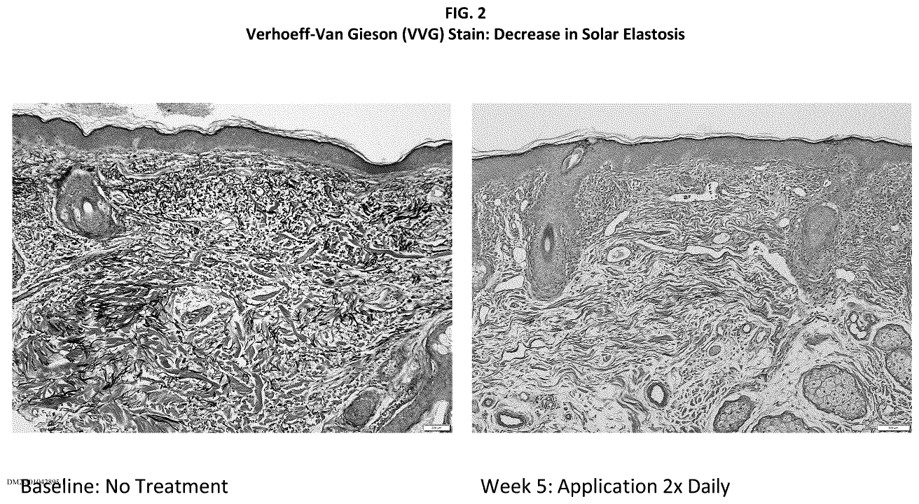

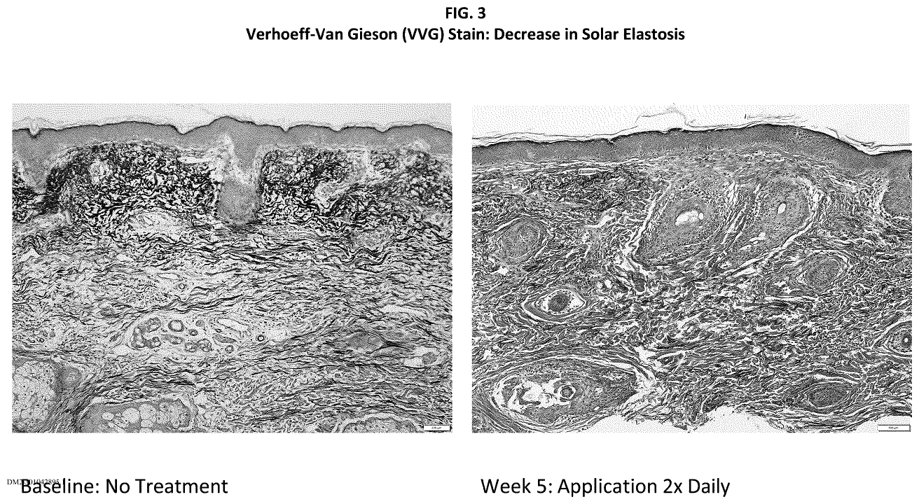

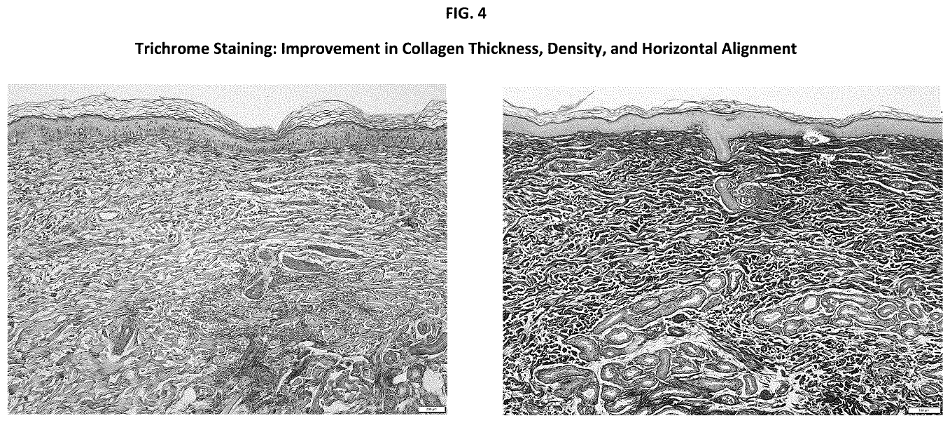

Collagen and Elastin Remodeling Trichrome staining revealed significant thickening, density, and horizontal alignment of collagen bundles in the superficial dermis. This improvement suggests that the novel compositions of the invention foster structured collagen synthesis, reinforcing dermal strength. Additionally, Verhoeff-Van Gieson staining (VVG) showed reduced solar elastosis, with less fiber fragmentation and clumping. These changes point to novel compositions' effectiveness in supporting both collagen and elastin production and integrity, contributing to firmer and more youthful dermal architecture. (See FIGS. 2, 3, and 4).

### Example 3

### Composition Analysis

### Scientific Validation & Analysis

The efficacy of the novel compositions described herein has been rigorously validated through third-party testing, providing strong evidence of its transformative potential.
a. High-Performance Liquid Chromatography-Mass Spectrometry (HPLC-MS): This technique offers unparalleled precision in identifying and quantifying the bioactive molecules, and it identified over 1,200+ growth factors, proteins, and peptides in the proprietary Human Mesenchymal Conditioned Media composition claimed herein.
b. Antibody Microarray: Measured high expression levels of key factors like PDGF, VEGF, EGF, etc. essential for wound-healing, anti-aging, skin regeneration, and reducing inflammation in the proprietary Human Mesenchymal Conditioned Media composition.
c. Nanoparticle Analysis and Electron Microscopy: Using nanoparticle tracking analysis (NTA) and electron microscopy, the concentration of exosomes in in the proprietary Human Mesenchymal Conditioned Media composition, was quantified at over 361+ billion exosomes per milliliter.

### Comprehensive Regenerative Pathways: A Molecular Approach to Skin Health

Protein pathway analysis using Reactome (www.reactome.org) revealed that the proprietary Human Mesenchymal Conditioned Media composition plays a pivotal role in several key skin regeneration pathways, including:
a. Hemostasis: in the proprietary Human Mesenchymal Conditioned Media composition's rich concentration of growth factors rapidly initiates the wound healing process, a critical function for both post-procedural recovery and age-related skin repair.
b. Extracellular Matrix Organization: Stimulating the production of collagen and elastin, the proprietary Human Mesenchymal Conditioned Media composition helps restore the structural integrity of the skin, promoting firmness, elasticity, and resilience.
c. Collagen Biosynthesis and Degradation: The proprietary Human Mesenchymal Conditioned Media composition, supports healthy collagen turnover, ensuring both the synthesis of new collagen fibers and the controlled breakdown of aged tissue, resulting in smoother, younger-looking skin.
d. Inflammation Modulation: The components of the proprietary Human Mesenchymal Conditioned Media composition, including syndecan-related proteins, reduce inflammation, accelerating recovery from procedures and promoting long-term skin health.

### Gene Expression and Cellular Senescence Modulation

The novel compositions herein further demonstrate transformative impact on skin health through the ability to influence gene expression associated with aging, inflammation, and skin structure. In independent studies on human dermal fibroblasts cultured under senescence-inducing conditions, the compositions effectively reversed the expression of key senescence markers and inflammatory cytokines, highlighting its anti-aging and regenerative potential.

Under these senescence-promoting conditions, genes like CDKN2A, IL6, and IGFBP3, linked to cell cycle interruption, chronic inflammation, and reduced growth factor activity, were significantly upregulated. The compositions comprising the proprietary Human Mesenchymal Conditioned Media, however, successfully downregulated these markers, restoring their expression to levels seen in healthy, non-senescent cells. This shift underscores the ability of the novel compositions and formulations described herein to counteract cellular aging and promote a more youthful skin environment.

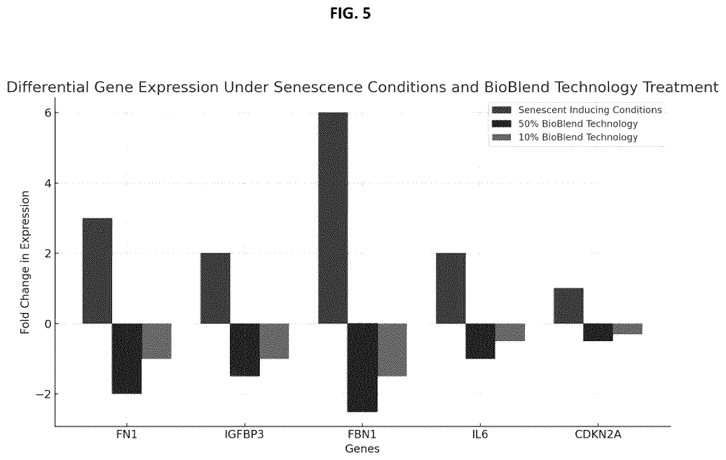

Additionally, the novel formulations claimed herein normalized the expression of structural proteins critical for skin integrity, such as fibronectin (FN1) and fibrillin-1 (FBN1). By maintaining the balance of these extracellular matrix components, the formulations support improved skin elasticity and resilience, contributing to visible enhancements in tone and firmness. (see FIG. 5)

### Selective Cellular Regeneration—Human Dermal Fibroblast vs Melanoma

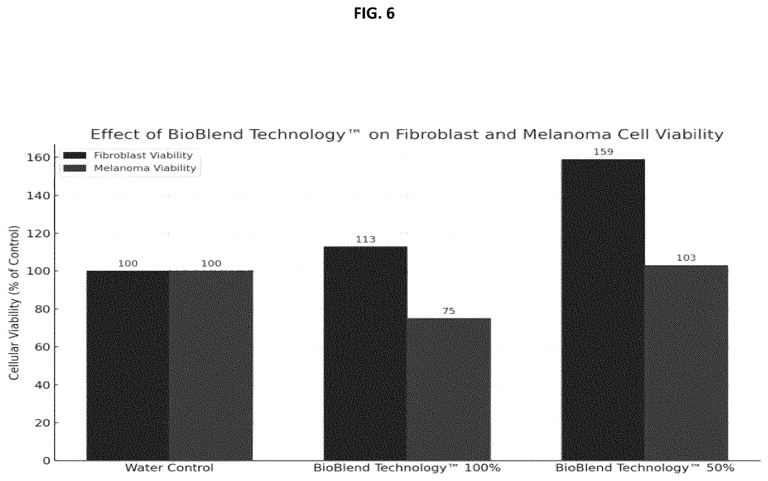

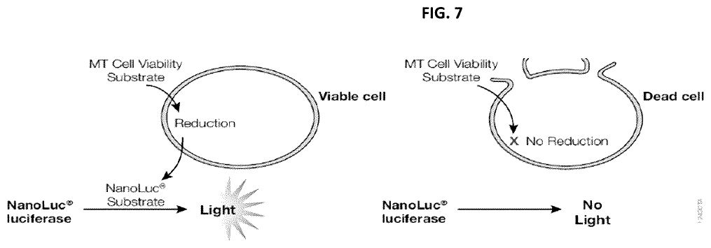

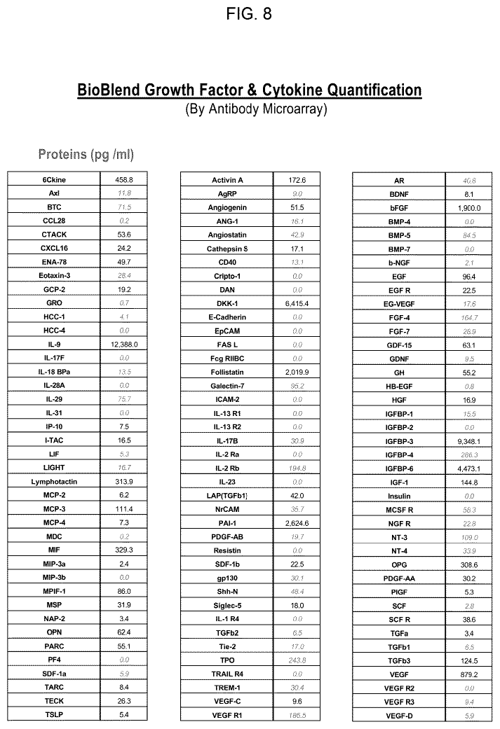

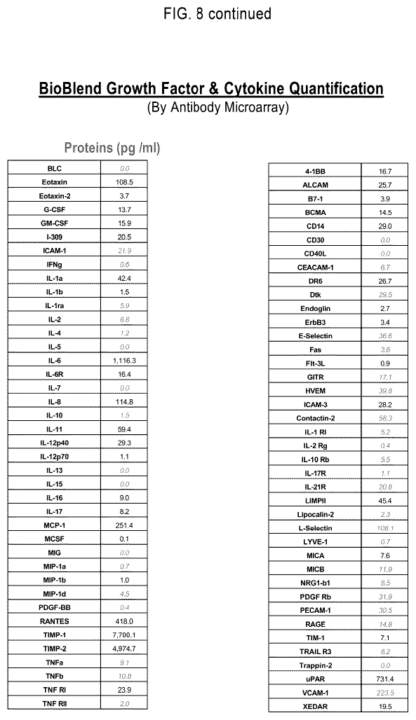

One of the unique advantages of utilizing the proprietary Human Mesenchymal Conditioned Media compositions is the ability to selectively promote healthy cellular regeneration. Using the RealTime-Glo™ MT Assay, the compositions/formulations were shown to increase the viability of normal human dermal fibroblasts by over 20%, while showing no proliferative effects on malignant melanoma cells. This precision in regulating cellular activity ensures the safety of the proprietary compositions and formulations in preventing unwanted cell growth, while effectively promoting regenerative pathways in healthy cells. (see FIG. 6)

### Example 4

### Formulations

### Composition A

TABLE 5
Composition A

| INGREDIENTS INCI/CTFA NAME | % W/W |
|---|---|
| Water (Aqua) | 61.073 |
| Sodium Hyaluronate | 0.200 |
| Pentylene Glycol | 3.000 |
| Trisodium Ethylenediamine Disuccinate | 0.300 |
| Water (Aqua) | |
| Niacinamide | 3.000 |
| Propanediol | 0.300 |
| Lecithin | |
| Polyacrylate-13 | 3.000 |
| Polyisobutene | |
| Polysorbate 20 | |
| Cetyl Stearate | 0.500 |
| Helianthus Annuus (Sunflower) Seed Oil | 0.020 |
| Arnica Montana Flower Extract | |
| Tetrahexyldecyl Ascorbate | 0.100 |
| Dimethyl Isosorbide | 2.000 |
| Cetyl Ethylhexanoate | 3.000 |
| Human Mesenchymal Conditioned Media | 12.000 |
| (96.05%) | |
| Glycerin (3%) | |
| Phenoxyethanol (0.75%) | |
| Ethylhexylglycerin (0.2%) | |
| Glycerin (99.98%) | 5.000 |
| Tetrapeptide-14 (0.02%) | |
| Water (Aqua) | 2.000 |
| Palmitoyl Tripeptide-1 | 0.001 |
| Carnosine | 0.200 |
| Water (64.92%) | 0.010 |
| Ammonium Sulfate (stabilizer) (34.92%) | |
| sh-Polypeptide-77 (0.04%) | |
| sh-Polypeptide-2 (0.04%) | |
| Superoxide Dismutase (0.08%) | |
| sh -Polypeptide-3 | 0.010 |
| sr-Honey Bee Defensin-1/Royal Jelly | 0.050 |
| Extract | |
| Water 80.475% | 3.000 |
| Lecithin 5% | |
| Acetyl Glutamine 5% | |
| Propanediol 5.5% | |
| sh-Oligopeptide-1 0.005% | |
| sh-Oligopeptide-2 0.005% | |
| sh-Polypeptide-1 0.005% | |
| sh-Polypeptide-9 0.005% | |
| sh-Polypeptide-11 0.005% | |
| Bacillus/folic ferment filtrate extract | |
| 1.00% | |
| Sodium hyaluronate 1.00% | |
| Caprylyl glycol 1.00% | |
| Ethyhexylglycerin 1.00% | |
| Dehydroacetic Acid 7.99635% | 1.000 |
| Benzyl Alcohol (fragrance allergen | |
| intentionally added) 87.99635%. | |
| Water 4% | |
| Toluene (trace impurity) 0.0056% | |
| Hydroquinone (trace impurity) 0.0017% | |
| Benzaldehyde (fragrance allergen) | |
| 0.0356% | |
| Citric Acid | 0.236 |
| Sodium Hydroxide | 0.000 |
| | 100.00 |

### Composition B

TABLE 6
Composition B

| INGREDIENTS INCI/CTFA NAME | % W/W |
|---|---|
| Water (Aqua) | 65.7473 |
| Sodium Hyaluronate | 0.1500 |
| Glycerin | 0.5000 |
| Propanediol 74.97% | 0.1000 |
| Lecithin 24.97% | |
| Tocopherol 0.04% | |
| Helianthus Annuus (Sunflower) Seed Oil | |
| 0.02% | |
| Helianthus Annuus (Sunflower) Seed Oil | 0.0200 |
| 70% | |
| Arnica Montana Flower Extract 30% | |
| Eugenol (fragrance allergen) 0.05% | |
| beta-Caryophyllene (fragrance allergen) | |
| 0.1% | |
| Trisodium Ethylenediamine Disuccinate | 0.3000 |
| Water (Aqua) | |
| Dehydroacetic Acid | 1.000 |
| Benzyl Alcohol | |
| Pentylene Glycol | 4.000 |
| Niacinamide | 3.000 |
| sh-Oligopeptide-1 | 0.0100 |
| sh -Polypeptide-3 | 0.0100 |
| sr-Honey Bee Defensin-1/Royal Jelly | 0.0500 |
| Extract | |
| Palmitoyl Hexapeptide | 0.0010 |
| Acetyl Tetrapeptide-2 | 0.0050 |
| Water | 3.000 |
| Lecithin | |
| Acetyl Glutamine | |
| Propanediol | |
| sh-Oligopeptide-1 | |
| sh-Oligopeptide-2 | |
| sh-Polypeptide-1 | |
| sh-Polypeptide-9 | |
| sh-Polypeptide-11 | |
| Bacillus/folic ferment filtrate extract | |
| Sodium hyaluronate | |
| Caprylyl glycol | |
| Ethyhexylglycerin | |
| Glycerin | 5.000 |
| Tetrapeptide-14 | |
| Glycerin 70% | 2.000 |
| Water 28% | |
| Tasmania Lanceolata Fruit Leaf extract | |
| 1.95% | |
| Pectinase (pectic enzyme) 0.05% | |
| Eugenol (fragrance allergen) 0.0002% | |
| Linalool (fragrance allergen) 0.0026% | |
| Human Mesenchymal Conditioned Media | 15.000 |
| 96.05% | |
| Phenoxyethanol 0.75% | |
| Ethylhexylglycerin 0.2% | |
| Glycerin 3% | |
| Citric Acid | 0.1067 |
| | 100.0000 |

### Composition C

TABLE 7
Composition C

| INGREDIENTS INCI/CTFA NAME | % W/W |
|---|---|
| Water (Aqua) | 56.920 |
| Glycerin | 1.000 |
| Sodium Hyaluronate | 0.150 |
| Propanediol | 0.100 |
| Lecithin | |
| Helianthus Annuus (Sunflower) Seed Oil 70% | 0.020 |
| Arnica Montana Flower Extract 30% | |
| Eugenol (fragrance allergen) 0.05% | |
| beta-Caryophyllene (fragrance allergen) 0.1% | |
| Trisodium Ethylenediamine Disuccinate | 0.300 |
| Dehydroacetic Acid 7.99635% | 1.000 |
| Benzyl Alcohol (fragrance allergen | |
| intentionally added) 87.99635%. | |
| Water 4% | |
| Toluene (trace impurity) 0.0056% | |
| Hydroquinone (trace impurity) 0.0017% | |
| Benzaldehyde (fragrance allergen) 0.0356% | |
| Pentylene Glycol | 4.000 |
| Tranexamic Acid | 8.500 |
| Niacinamide | 3.000 |
| Acetyl Glycyl Beta-Alanine | 1.000 |
| Water (Aqua) 97.5% | 2.000 |
| 1,2-Hexanediol 2% | |
| Pentasodium Tetracarboxymethyl Dipeptide- | |
| 51 0.125% | |
| Pentasodium Tetracarboxymethyl | |
| Acetylhydroxyprolyl Dipeptide-12 0.375% | |
| sr-Honey Bee Defensin-1 | 0.010 |
| Royal Jelly Extract | |
| Glycerin | 2.000 |
| Water | |
| Tasmannia Lanceolata Fruit/Leaf Extract | |
| Glycerin | 5.000 |
| Tetrapeptide-14 | |
| Human Mesenchymal Conditioned Media | 15.000 |
| 96.05% | |
| Phenoxyethanol 0.75% | |
| Ethylhexylglycerin 0.2% | |
| Glycerin 3% | |
| Citric Acid | 0.000 |
| Sodium Hydroxide | 0.000 |
| | 100.000 |

### Composition D

TABLE 8
Composition D

| INGREDIENTS INCI/CTFA NAME | % W/W |
|---|---|
| Water (Aqua) | 50.5030 |
| Sodium Hyaluronate | 0.1200 |
| Propanediol | 0.1000 |
| Lecithin | |
| Trisodium Ethylenediamine | 0.3000 |
| Disuccinate | |
| Dehydroacetic Acid | 1.0000 |
| Benzyl Alcohol | |
| Pentylene Glycol | 4.0000 |
| Caffeine | 1.0000 |
| Glycerin | 1.0000 |
| Water (Aqua) | |
| Serenoa Serrulata Fruit Extract | |
| sh-polypeptide-3 | 0.0500 |
| Glycerin 50% | 0.5000 |
| Water (Aqua) 48.50% | |
| Nasturtium officinale extract (Watercress) | |
| Extract 0.7% | |
| Tropaeolum majus (Garden Nasturtium) | |
| Extract 0.7% | |
| Sorbic Acid 0.10% | |
| Water | 3.0000 |
| Lecithin | |
| Acetyl Glutamine | |
| Propanediol | |
| sh-Oligopeptide-1 | |
| sh-Oligopeptide-2 | |
| sh-Polypeptide-1 | |
| sh-Polypeptide-9 | |
| sh-Polypeptide-11 | |
| Bacillus/folic acid ferment filtrate extract | |
| Sodium hyaluronate | |
| Caprylyl glycol | |
| Ethylhexylglycerin | |
| Water | 10.0000 |
| 1,2-Hexanediol | |
| Caprylyl Glycol | |
| Tripeptide-80 | |
| Acetyl Tetrapeptide-54 Amide | |
| Water | 10.0000 |
| 1,2 Hexanediol | |
| Caprylyl Glycol | |
| Tripeptide-80 | |
| Acetyl sh Tripeptide-4 Amide | |
| Human Mesenchymal Conditioned Media | 15.0000 |
| 96.05% | |
| Phenoxyethanol 0.75% | |
| Ethylhexylglycerin 0.2% | |
| Glycerin 3% | |
| Potassium Azeloyl Diglycinate | 3.0000 |
| Water | |
| Sodium Hydroxide | 0.181 |
| Citric Acid | 0.246 |
| | 100.0000 |

### Example 5

### Skin Repair Following Dog Bite

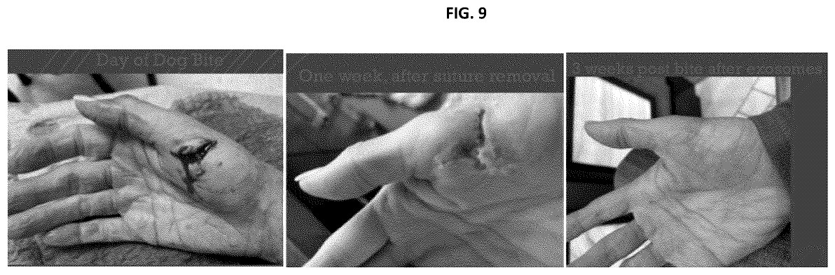

As shown in FIG. 9, following a serious dog bite an individual experienced severe laceration on the palm of her right hand. The individual's skin was severely damaged. Following the application of Composition B, scarring and other expected disfiguration was significantly diminished: a similar level of skin rejuvenation would not be expected had routine treatments been utilized instead.

Composition B applied daily over a three-week period, significantly accelerated the body's natural recovery process. According to a review of comparable cases, skin complications of this nature typically require 6-8 weeks to improve, whereas this individual's case demonstrated substantial rejuvenation in just 3 weeks as a result of using the unique Composition B solution.

1. A composition comprising Human Mesenchymal Conditioned Media from human-derived cell sources, wherein the human-derived cell sources comprise bone marrow mesenchymal cells (BM-MSCs), umbilical cord mesenchymal cells (UC-MSCs), and fibroblast cells,
wherein the BM-MSCs, UC-MSCs, and fibroblast cells are cultured in growth media specifically formulated for optimal cell proliferation and optimized for the secretion of regenerative molecules,
wherein the growth media for BM-MSCs comprises Dulbecco's Modified Eagle Medium-Ham's F-12 (DMEM-F12) supplemented with fetal bovine serum (FBS), and growth factors (rhFGF basic, IGF-1);
wherein the growth factors for UC-MSCs comprises Dulbecco's Modified Eagle Medium-Ham's F-12 (DMEM-F12) supplemented with fetal bovine serum (FBS), and growth factors (rhEGF, rhFGF basic, rhFGF acidic);
wherein the growth media for culturing fibroblast cells comprises Dulbecco's Modified Eagle Medium (DMEM) with 8% FBS.

2. The composition of claim 1, wherein growth media for BM-MSCs is supplemented with 10% fetal bovine serum (FBS), 15 ng/ml IGF-1, 125 pg/mL rhFGF basic, and 2.4 mM L-Alanyl-L-Glutamine.

3. The composition of claim 1, wherein growth media for culturing UC-MSCs is supplemented with 4% FBS, 5 ng/mL rhFGF basic, 5 ng/mL rhFGF acidic, 5 ng/mL rhEGF and 2.4 mM L-Alanyl-L-Glutamine.

4. The composition of claim 1, wherein the BM-MSCs, UC-MSCs, and fibroblast cells are maintained in the presence of antibiotic antimycotic solutions, wherein the antibiotic antimycotic solution comprises penicillin, streptomycin, amphotericin, or hygromycin; and
wherein certain embodiments of the antimycotic solution specifically comprise 50-200 Units/mL penicillin G, 50-200 ug/mL streptomycin and 5-50 ng/ml amphotericin, or
wherein certain embodiments of the antimycotic solution specifically comprise 100 Units/mL penicillin G, 100 ug/mL streptomycin and 25 ng/ml amphotericin, or
wherein certain embodiments of the antimycotic solution specifically comprise 1-50 ng/mL hygromycin, or
wherein certain embodiments of the antimycotic solution specifically comprise 10 ng/ml hygromycin.

5. The composition of claim 1, wherein the Human Mesenchymal Conditioned Media is treated with 1% preservative comprising 1% EUXYL® at room temperature when the Human Mesenchymal Conditioned Media is necessary for immediate use.

6. The composition of claim 1, wherein the Human Mesenchymal Conditioned Media is treated with 3% sterile glycerol for long-term storage and frozen −80° C. in low-temperature-resistant containers when the Human Mesenchymal Conditioned Media is necessary for long-term storage.

7. The composition of claim 1, wherein the composition is used to create formulations consisting of Compositions A, B, C and D.

8. The composition of claim 7, wherein the formulations further comprise additional ingredients comprising proteins, peptides, cytokines, growth factors, exosomes, solvents, hydrocarbon oils hydration compounds, humectants, penetration enhancers, chelators, thickening polymers, mineral thickening agents, non-mineral thickening agents, anti-inflammatory agents, antioxidants, skin regeneration agents, collagen, stabilizers, redness reduction agents, anti-aging components, anti-wrinkle components, anti-free radical agents, DNA repair enzymes, growth factors, non-volatile fatty substances, inorganic pigments, organic colorants, soft focus powder, water-soluble solvents, preservatives, fragrances, or salts.

9. The composition of claim 8, wherein the formulation comprises Composition A Formulation and wherein the Composition A Formulation consists of a high-efficacy facial serum enabling anti-aging benefits, targeting the appearance of fine lines and wrinkles, improvement of skin tone, and improvement of elasticity.

10. The composition of claim 9, wherein the Composition A Formulation comprises Water (Aqua), Human Mesenchymal Human Mesenchymal Conditioned Media, Glycerin, Pentylene Glycol, Niacinamide, Cetyl Ethylhexanoate, Dimethyl Isosorbide, Polyacrylate-13, sh-Polypeptide-3, sh-Polypeptide-77, sh-Polypeptide-2, sr-Honey Bee Defensin-1, Palmitoyl Tripeptide-1, Tetrapeptide-14, sh-Oligopeptide-2, sh-Polypeptide-1, sh-Polypeptide-9, sh-Polypeptide-11, sh-Oligopeptide-1, Superoxide Dismutase, Carnosine, Cetyl Stearate, Polyisobutene, Polysorbate 20, Tetrahexyldecyl Ascorbate, Propanediol, Lecithin, Sodium Hyaluronate, Helianthus annuus (Sunflower) Seed Oil, Arnica montana Flower Extract, Acetyl Glutamine, Bacillus/Folic Acid Ferment Filtrate Extract, Caprylyl Glycol, Ethylhexylglycerin, Trisodium Ethylenediamine Disuccinate, Dehydroacetic Acid, Phenoxyethanol, Ammonium Sulfate, Tocopherol, Benzyl Alcohol, Citric Acid, and Sodium Hydroxide.

11. The composition of claim 9, wherein the Composition A Formulation comprises Water (Aqua) 50-70 (% w/w), Human Mesenchymal Human Mesenchymal Conditioned Media 5 (% w/w), Glycerin 1-10 (% w/w), Pentylene Glycol 1-5 (% w/w), Niacinamide 1-10 (% w/w), Cetyl Ethylhexanoate 0.1-10.0 (% w/w), Dimethyl Isosorbide 0.5-5.0 (% w/w), Polyacrylate-13 0.1-5 (% w/w), sr-Honey Bee Defensin-1 0.001-0.1 (% w/w), Palmitoyl Tripeptide-1 0.001-0.01 (% w/w), Tetrapeptide-14 1-10 (% w/w), sh-Oligopeptide-2 0.1-1.0 (% w/w), Polypeptides (sh-Polypeptide-77 and sh-Polypeptide-2 and Superoxide Dismutase, sh-0.001-0.1 (% w/w)), sh-Polypeptide-3 0.001-0.1 (% w/w), sh-Oligopeptide-1 0.1-1.0 (% w/w), Carnosine 0.1-1.0 (% w/w), Cetyl Stearate 0.1-5.0 (% w/w), Polyisobutene 1-5 (% w/w), Polysorbate 20 1-5 (% w/w), Tetrahexyldecyl Ascorbate 0.01-2 (% w/w), Propanediol 0.1-5 (% w/w), Lecithin 0.1-5 (% w/w), Sodium Hyaluronate 0.1-1.0 (% w/w), Helianthus annuus (Sunflower) Seed Oil 0.01-2 (% w/w), Arnica montana Flower Extract 0.01-2 (% w/w), Acetyl Glutamine 0.1-5.0 (% w/w), Bacillus/Folic Acid Ferment Filtrate Extract 0.1-1.0 (% w/w), Caprylyl Glycol 0.1-5.0 (% w/w), Ethylhexylglycerin 0.1-5.0 (% w/w), Trisodium Ethylenediamine Disuccinate 0.1-1 (% w/w), Dehydroacetic Acid 0.1-5.0 (% w/w), Phenoxyethanol 0.1-5.0 (% w/w), Ammonium Sulfate 0.1-5.0 (% w/w), Tocopherol 0.1-5.0 (% w/w), Benzyl Alcohol 0.1-10.0 (% w/w), Citric Acid 0.1-10.0 (% w/w), and Sodium Hydroxide 0.0-5.0 (% w/w).

12. The composition of claim 9, wherein the Composition A Formulation comprises:
Composition A Formulation

| INGREDIENTS INCI/CTFA NAME | % W/W |
|---|---|
| Water (Aqua) | 61.073 |
| Sodium Hyaluronate | 0.200 |
| Pentylene Glycol | 3.000 |
| Trisodium Ethylenediamine Disuccinate | 0.300 |
| Water (Aqua) | |
| Niacinamide | 3.000 |
| Propanediol | 0.300 |
| Lecithin | |
| Polyacrylate-13 | 3.000 |
| Polyisobutene | |
| Polysorbate 20 | |
| Cetyl Stearate | 0.500 |
| Helianthus Annuus (Sunflower) Seed Oil | 0.020 |
| Arnica Montana Flower Extract | |
| Tetrahexyldecyl Ascorbate | 0.100 |
| Dimethyl Isosorbide | 2.000 |
| Cetyl Ethylhexanoate | 3.000 |
| Human Mesenchymal Conditioned Media | 12.000 |
| (96.05%) | |
| Glycerin (3%) | |
| Phenoxyethanol (0.75%) | |
| Ethylhexylglycerin (0.2%) | |
| Glycerin (99.98%) | 5.000 |
| Tetrapeptide-14 (0.02%) | |
| Water (Aqua) | 2.000 |
| Palmitoyl Tripeptide-1 | 0.001 |
| Carnosine | 0.200 |
| Water (64.92%) | 0.010 |
| Ammonium Sulfate (stabilizer) (34.92%) | |
| sh-Polypeptide-77 (0.04%) | |
| sh-Polypeptide-2 (0.04%) | |
| Superoxide Dismutase (0.08%) | |
| sh -Polypeptide-3 | 0.010 |
| sr-Honey Bee Defensin-1/Royal Jelly | 0.050 |
| Extract | |
| Water 80.475% | 3.000 |
| Lecithin 5% | |
| Acetyl Glutamine 5% | |
| Propanediol 5.5% | |
| sh-Oligopeptide-1 0.005% | |
| sh-Oligopeptide-2 0.005% | |
| sh-Polypeptide-1 0.005% | |
| sh-Polypeptide-9 0.005% | |
| sh-Polypeptide-11 0.005% | |
| Bacillus/folic ferment filtrate extract | |
| 1.00% | |
| Sodium hyaluronate 1.00% | |
| Caprylyl glycol 1.00% | |
| Ethyhexylglycerin 1.00% | |
| Dehydroacetic Acid 7.99635% | 1.000 |
| Benzyl Alcohol (fragrance allergen | |
| intentionally added) 87.99635%. | |
| Water 4% | |
| Toluene (trace impurity) 0.0056% | |
| Hydroquinone (trace impurity) 0.0017% | |
| Benzaldehyde (fragrance allergen) | |
| 0.0356% | |
| Citric Acid | 0.236 |
| Sodium Hydroxide | 0.000 |
| | 100.00 |

13. The composition of claim 8, wherein the formulation comprises a Composition B Formulation and wherein Composition B Formulation promotes overall skin rejuvenation, supports collagen and elastin production, improves the appearance of aging skin, enhances skin smoothness and luminosity, and provides calming and soothing benefits to the skin, and wherein the Composition B Formulation comprises Water (Aqua), Human Mesenchymal Human Mesenchymal Conditioned Media, Glycerin, Pentylene Glycol, Niacinamide, sh-Oligopeptide-1, sh-Polypeptide-3, sr-Honey Bee Defensin-1, Acetyl Tetrapeptide-2, Palmitoyl Hexapeptide-12, Tetrapeptide-14, sh-Oligopeptide-2, sh-Polypeptide-1, sh-Polypeptide-9, sh-Polypeptide-11, Propanediol, Lecithin, Sodium Hyaluronate, Helianthus annuus (Sunflower) Seed Oil, Arnica montana Flower Extract, Tasmannia lanceolata Fruit/Leaf Extract, Acetyl Glutamine, Bacillus/Folic Acid Ferment Filtrate Extract, Caprylyl Glycol, Ethylhexylglycerin, Trisodium Ethylenediamine Disuccinate, Dehydroacetic Acid, Phenoxyethanol, Ammonium Sulfate, Tocopherol, Benzyl Alcohol, Citric Acid, Sodium Hydroxide.

14. The composition of claim 8, wherein the formulation comprises a Composition B Formulation and wherein the Composition B Formulation comprises Water (Aqua) 40-80 (% w/w), Human Mesenchymal Human Mesenchymal Conditioned Media 5-30 (% w/w), Glycerin 1-15 (% w/w), Pentylene Glycol 1-10 (% w/w), Niacinamide 1-15 (% w/w), sh-Oligopeptide-1 1-10 (% w/w), sh-Polypeptide-3 0.001-1 (% w/w), sr-Honey Bee Defensin-1 0.001-1 (% w/w), Acetyl Tetrapeptide-2, Palmitoyl Hexapeptide-12 0.001-1 (% w/w), Tetrapeptide-14 0.001-1 (% w/w), sh-Oligopeptide-2 0.001-5 (% w/w), sh-Polypeptide-1 0.001-5 (% w/w), sh-Polypeptide-9 0.001-5 (% w/w), sh-Polypeptide-11 0.001-5 (% w/w), Propanediol 0.001-5 (% w/w), Lecithin 0.001-5 (% w/w), Sodium Hyaluronate 0.001-5 (% w/w), Helianthus annuus (Sunflower) Seed Oil 0.001-5 (% w/w), Arnica montana Flower Extract 0.001-5 (% w/w), Tasmannia lanceolata Fruit/Leaf Extract 0.001-5 (% w/w), Acetyl Glutamine 0.001-5 (% w/w), Bacillus/Folic Acid Ferment Filtrate Extract 0.001-5 (% w/w), Caprylyl Glycol 0.001-5 (% w/w), Ethylhexylglycerin 0.001-5 (% w/w), Trisodium Ethylenediamine Disuccinate 0.001-5 (% w/w), Dehydroacetic Acid 0.001-5 (% w/w), Phenoxyethanol 0.001-20 (% w/w), Ammonium Sulfate 0.001-10 (% w/w), Tocopherol 0.001-10 (% w/w), Benzyl Alcohol 0.001-10 (% w/w), Citric Acid 0.1-10.0 (% w/w), and Sodium Hydroxide 0.0-5.0 (% w/w).

15. The composition of claim 14, wherein the Composition B Formulation comprises:
Composition B Formulation

| INGREDIENTS INCI/CTFA NAME | % W/W |
|---|---|
| Water (Aqua) | 65.7473 |
| Sodium Hyaluronate | 0.1500 |
| Glycerin | 0.5000 |
| Propanediol 74.97% | 0.1000 |
| Lecithin 24.97% | |
| Tocopherol 0.04% | |
| Helianthus Annuus (Sunflower) Seed Oil | |
| 0.02% | |
| Helianthus Annuus (Sunflower) Seed Oil | 0.0200 |
| 70% | |
| Arnica Montana Flower Extract 30% | |
| Eugenol (fragrance allergen) 0.05% | |
| beta-Caryophyllene (fragrance allergen) | |
| 0.1% | |
| Trisodium Ethylenediamine Disuccinate | 0.3000 |
| Water (Aqua) | |
| Dehydroacetic Acid | 1.000 |
| Benzyl Alcohol | |
| Pentylene Glycol | 4.000 |
| Niacinamide | 3.000 |
| sh-Oligopeptide-1 | 0.0100 |
| sh -Polypeptide-3 | 0.0100 |
| sr-Honey Bee Defensin-1/Royal Jelly | 0.0500 |
| Extract | |
| Palmitoyl Hexapeptide | 0.0010 |
| Acetyl Tetrapeptide-2 | 0.0050 |
| Water | 3.000 |
| Lecithin | |
| Acetyl Glutamine | |
| Propanediol | |
| sh-Oligopeptide-1 | |
| sh-Oligopeptide-2 | |
| sh-Polypeptide-1 | |
| sh-Polypeptide-9 | |
| sh-Polypeptide-11 | |
| Bacillus/folic ferment filtrate extract | |
| Sodium hyaluronate | |
| Caprylyl glycol | |
| Ethyhexylglycerin | |
| Glycerin | 5.000 |
| Tetrapeptide-14 | |
| Glycerin 70% | 2.000 |
| Water 28% | |
| Tasmania Lanceolata Fruit Leaf extract | |
| 1.95% | |
| Pectinase (pectic enzyme) 0.05% | |
| Eugenol (fragrance allergen) 0.0002% | |
| Linalool (fragrance allergen) 0.0026% | |
| Human Mesenchymal Conditioned Media | 15.000 |
| 96.05% | |
| Phenoxyethanol 0.75% | |
| Ethylhexylglycerin 0.2% | |
| Glycerin 3% | |
| Citric Acid | 0.1067 |
| | 100.0000 |

16. The composition of claim 8, wherein the formulation comprises a Composition C Formulation, and wherein Composition C Formulation addresses pigmentation issues, reduces discoloration, improves skin tone, reduces uneven skin tone, and alleviates sun damage; and wherein the Composition C Formulation comprises Water (Aqua), Human Mesenchymal Human Mesenchymal Conditioned Media, Tranexamic Acid, Glycerin, Pentylene Glycol, Niacinamide, Acetyl Glycyl Beta-Alanine, 1,2-Hexanediol, Pentasodium Tetracarboxymethyl Acetylhydroxyprolyl Dipeptide-12, Pentasodium Tetracarboxymethyl Dipeptide-51, Tetrapeptide-14, sr-Honey Bee Defensin-1, Propanediol, Lecithin, Sodium Hyaluronate, Helianthus annuus (Sunflower) Seed Oil, Arnica montana Flower Extract, Tasmannia lanceolata Fruit/Leaf Extract, Ethylhexylglycerin, Trisodium Ethylenediamine Disuccinate, Dehydroacetic Acid, Phenoxyethanol, Ammonium Sulfate, Tocopherol, Benzyl Alcohol, Citric Acid, Sodium Hydroxide.

17. The composition of claim 16, wherein the Composition C Formulation comprises Water (Aqua) 40-80 (% w/w), Human Mesenchymal Human Mesenchymal Conditioned Media 5-30 (% w/w), Tranexamic Acid 1-15 (% w/w), Glycerin 0.001-5 (% w/w), Pentylene Glycol 0.01-15 (% w/w), Niacinamide 0.1-10 (% w/w), Acetyl Glycyl Beta-Alanine 0.1-10 (% w/w), 1,2-Hexanediol 0.001-5 (% w/w), Pentasodium Tetracarboxymethyl Acetylhydroxyprolyl Dipeptide-12 0.001-5 (% w/w), Pentasodium Tetracarboxymethyl Dipeptide-51 0.001-5 (% w/w), Tetrapeptide-14 0.001-5 (% w/w), sr-Honey Bee Defensin-1 0.001-5 (% w/w), Propanediol 0.001-5 (% w/w), Lecithin 0.001-5 (% w/w), Sodium Hyaluronate 0.001-5 (% w/w), Helianthus annuus (Sunflower) Seed Oil 0.001-5 (% w/w), Arnica montana Flower Extract 0.001-5 (% w/w), Tasmannia lanceolata Fruit/Leaf Extract 0.001-5 (% w/w), Ethylhexylglycerin 0.1-25 (% w/w), Trisodium Ethylenediamine Disuccinate 0.001-5 (% w/w), Dehydroacetic Acid 0.001-5 (% w/w), Phenoxyethanol 0.001-20 (% w/w), Ammonium Sulfate 0.001-10 (% w/w), Tocopherol 0.001-10 (% w/w), Benzyl Alcohol 0.001-10 (% w/w), Citric Acid 0.1-10.0 (% w/w), and Sodium Hydroxide 0.0-5.0 (% w/w).

18. The composition of claim 8, wherein the Composition C Formulation comprises:
Composition C Formulation

| INGREDIENTS INCI/CTFA NAME | % W/W |
|---|---|
| Water (Aqua) | 56.920 |
| Glycerin | 1.000 |
| Sodium Hyaluronate | 0.150 |
| Propanediol | 0.100 |
| Lecithin | |
| Helianthus Annuus (Sunflower) Seed Oil 70% | 0.020 |
| Arnica Montana Flower Extract 30% | |
| Eugenol (fragrance allergen) 0.05% | |
| beta-Caryophyllene (fragrance allergen) 0.1% | |
| Trisodium Ethylenediamine Disuccinate | 0.300 |
| Dehydroacetic Acid 7.99635% | 1.000 |
| Benzyl Alcohol (fragrance allergen | |
| intentionally added) 87.99635%. | |
| Water 4% | |
| Toluene (trace impurity) 0.0056% | |
| Hydroquinone (trace impurity) 0.0017% | |
| Benzaldehyde (fragrance allergen) 0.0356% | |
| Pentylene Glycol | 4.000 |
| Tranexamic Acid | 8.500 |
| Niacinamide | 3.000 |
| Acetyl Glycyl Beta-Alanine | 1.000 |
| Water (Aqua) 97.5% | 2.000 |
| 1,2-Hexanediol 2% | |
| Pentasodium Tetracarboxymethyl Dipeptide- | |
| 51 0.125% | |
| Pentasodium Tetracarboxymethyl | |
| Acetylhydroxyprolyl Dipeptide-12 0.375% | |
| sr-Honey Bee Defensin-1 | 0.010 |
| Royal Jelly Extract | |
| Glycerin | 2.000 |
| Water | |
| Tasmannia Lanceolata Fruit/Leaf Extract | |
| Glycerin | 5.000 |
| Tetrapeptide-14 | |
| Human Mesenchymal Conditioned Media | 15.000 |
| 96.05% | |
| Phenoxyethanol 0.75% | |
| Ethylhexylglycerin 0.2% | |
| Glycerin 3% | |
| Citric Acid | 0.000 |
| Sodium Hydroxide | 0.000 |
| | 100.000 |

19. The composition of claim 8, wherein the formulation comprises a Composition D Formulation, and wherein the Composition D Formulation stimulates hair follicles and promotes hair restoration, reduces thinning hair, reduces hair loss, improves scalp health, and inhibits the action of chemicals and hormones leading to hair loss, and wherein the Composition D Formulation comprises Water (Aqua), Human Mesenchymal Human Mesenchymal Conditioned Media, Pentylene Glycol, Glycerin, Caffeine, 1,2-Hexanediol, Acetyl Tripeptide-54 Amide, Acetyl sh-Tripeptide-4 Amide, Tripeptide-80, sh-Polypeptide-3, Acetyl Glutamine, sh-Oligopeptide-1, sh-Oligopeptide-2, sh-Polypeptide-1, sh-Polypeptide-9, sh-Polypeptide-11, Propanediol, Lecithin, Sodium Hyaluronate, Helianthus annuus (Sunflower) Seed Oil, Nasturtium officinale (Watercress) Extract, Tropaeolum majus (Garden Nasturtium) Extract, Sorbic Acid, Potassium Azeloyl Diglycinate, Extract, Serenoa repens (Saw Palmetto) Berry Extract, Bacillus/Folic Acid Ferment Filtrate Extract, Caprylyl Glycol, Ethylhexylglycerin, Trisodium Ethylenediamine Disuccinate, Dehydroacetic Acid, Phenoxyethanol, Tocopherol, Benzyl Alcohol, Citric Acid, Sodium Hydroxide.

20. The composition of claim 19, wherein the Composition D Formulation comprises Water (Aqua) 40-80 (% w/w), Human Mesenchymal Human Mesenchymal Conditioned Media 5-25 (% w/w), Pentylene Glycol 0.1-10 (% w/w), Glycerin 0.1-5 (% w/w), Caffeine 0.1-10 (% w/w), 1,2-Hexanediol 0.01-20 (% w/w), Acetyl Tripeptide-54 Amide 0.001-25 (% w/w), Acetyl sh-Tripeptide-4 Amide 0.001-25 (% w/w), Tripeptide-80 0.001-25 (% w/w), sh-Polypeptide-3 0.001-25 (% w/w), Acetyl Glutamine 0.001-10 (% w/w), sh-Oligopeptide-1 0.001-10 (% w/w), sh-Oligopeptide-2 0.001-10 (% w/w), sh-Polypeptide-1 0.001-10 (% w/w), sh-Polypeptide-9 0.001-10 (% w/w), sh-Polypeptide-11 0.001-10 (% w/w), Propanediol 0.001-10 (% w/w), Lecithin 0.001-10 (% w/w), Sodium Hyaluronate 0.001-10 (% w/w), Helianthus annuus (Sunflower) Seed Oil 0.001-5 (% w/w), Nasturtium officinale (Watercress) Extract 0.001-5 (% w/w), Tropaeolum majus (Garden Nasturtium) Extract 0.001-5 (% w/w), Sorbic Acid 0.001-10 (% w/w), Potassium Azeloyl Diglycinate 0.001-5 (% w/w), Extract, Serenoa repens (Saw Palmetto) Berry Extract 0.001-5 (% w/w), Bacillus/Folic Acid Ferment Filtrate Extract 0.001-5 (% w/w), Caprylyl Glycol 0.001-5 (% w/w), Ethylhexylglycerin 0.1-25 (% w/w), Trisodium Ethylenediamine Disuccinate 0.001-5 (% w/w), Dehydroacetic Acid 0.001-5 (% w/w), Phenoxyethanol 0.001-20 (% w/w), Tocopherol 0.001-10 (% w/w), Benzyl Alcohol 0.001-10 (% w/w), Citric Acid 0.1-10.0 (% w/w), and Sodium Hydroxide 0.0-5.0 (% w/w).

21. The composition of claim 8, wherein the Composition D Formulation comprises:
Composition D Formulation

| INGREDIENTS INCI/CTFA NAME | % W/W |
|---|---|
| Water (Aqua) | 50.5030 |
| Sodium Hyaluronate | 0.1200 |
| Propanediol | 0.1000 |
| Lecithin | |
| Trisodium Ethylenediamine | 0.3000 |
| Disuccinate | |
| Dehydroacetic Acid | 1.0000 |
| Benzyl Alcohol | |
| Pentylene Glycol | 4.0000 |
| Caffeine | 1.0000 |
| Glycerin | 1.0000 |
| Water (Aqua) | |
| Serenoa Serrulata Fruit Extract | |
| sh-polypeptide-3 | 0.0500 |
| Glycerin 50% | 0.5000 |
| Water (Aqua) 48.50% | |
| Nasturtium officinale extract (Watercress) | |
| Extract 0.7% | |
| Tropaeolum majus (Garden Nasturtium) | |
| Extract 0.7% | |
| Sorbic Acid 0.10% | |
| Water | 3.0000 |
| Lecithin | |
| Acetyl Glutamine | |
| Propanediol | |
| sh-Oligopeptide-1 | |
| sh-Oligopeptide-2 | |
| sh-Polypeptide-1 | |
| sh-Polypeptide-9 | |
| sh-Polypeptide-11 | |
| Bacillus/folic acid ferment filtrate extract | |
| Sodium hyaluronate | |
| Caprylyl glycol | |
| Ethylhexylglycerin | |
| Water | 10.0000 |
| 1,2-Hexanediol | |
| Caprylyl Glycol | |
| Tripeptide-80 | |
| Acetyl Tetrapeptide-54 Amide | |
| Water | 10.0000 |
| 1,2 Hexanediol | |
| Caprylyl Glycol | |
| Tripeptide-80 | |
| Acetyl sh Tripeptide-4 Amide | |
| Human Mesenchymal Conditioned Media | 15.0000 |
| 96.05% | |
| Phenoxyethanol 0.75% | |
| Ethylhexylglycerin 0.2% | |
| Glycerin 3% | |
| Potassium Azeloyl Diglycinate | 3.0000 |
| Water | |
| Sodium Hydroxide | 0.181 |
| Citric Acid | 0.246 |
| | 100.0000 |

22. A method for cosmetically improving skin conditions comprising:
utilizing novel formulations consisting of Composition A, B, C or D Formulations,
wherein the formulations comprise Human Mesenchymal Conditioned Media,
wherein the Human Mesenchymal Conditioned Media is obtained from human-derived cell sources comprising BM-MSCs, UC-MSCs, and fibroblast cells,
wherein the BM-MSCs, UC-MSCs, and fibroblast cells are cultured under unique conditions comprising the use of proprietary growth media,
wherein the proprietary growth media is specifically formulated for optimal cell proliferation and optimized for the secretion of regenerative molecules, and
monitoring the dermatological condition for improvement.

23. The method of claim 22, wherein the skin conditions include, but are not limited to, the appearance of wrinkles and fine lines, uneven tone, discoloration, roughness, dryness, and dull-looking skin.
